(19) United States
(12) Patent Application Publication
(10) Pub. No.: US 2014/0248289 A1
(43) Pub. Date: Sep. 4, 2014

(54) Methods and Compositions for the Treatment of Respiratory Conditions Via NKG2D Inhibition

(71) Applicant: University of Cincinnati, (US)

(72) Inventors: David Raulet, Berkeley, CA (US); Oliver Haworth, Berkeley, CA (US); Richard Locksley, Berkeley, CA (US); Michael Borchers, Cincinnati, OH (US)

(73) Assignees: UNIVERSITY OF CINCINNATI, Cincinnati, CA (US); THE REGENTS OF THE UNIVERSITY OF CALIFORNIA, Oakland, CA (US)

(21) Appl. No.: 14/240,732

(22) PCT Filed: Aug. 24, 2012
(86) PCT No.: PCT/US2012/052369

§ 371 Date: May 16, 2014

Related U.S. Application Data
(60) Provisional application No. 61/527,736, filed on Aug. 26, 2011.
(60) Provisional application No. 61/648,528, filed on May 17, 2012.
(60) Provisional application No. 61/606,701, filed on Mar. 5, 2012.

(51) Int. Cl. C07K 16/28; A61K 39/395; A61K 45/06

(52) U.S. Cl. CPC C07K 16/28; A61K 45/06; A61K 39/3955
USPC 424/173.1

(57) ABSTRACT

Methods and compositions for the treatment of respiratory conditions are provided. Aspects of the subject methods include administering to the subject a composition comprising an inhibitor of NKG2D-mediated activation of leukocytes. Also provided are compositions suitable for use in the subject methods, as well as pharmaceutical preparations thereof.

### CROSS-REFERENCE TO RELATED APPLICATIONS

This application claims the benefit of U.S. Provisional Patent Application No. 61/527,736, filed Aug. 26, 2011; U.S. Provisional Patent Application No. 61/606,701, filed Mar. 5, 2012; and U.S. Provisional Patent Application No. 61/648,528, filed May 17, 2012; the disclosures of which are incorporated herein by reference in their entirety.

### STATEMENT REGARDING FEDERALLY SPONSORED RESEARCH

This invention was made with Government support under Federal Grant Nos. P30ES006096; R01AI039642; R01CA093678 and R01ES015036 awarded by the National Institutes of Health. The Government has certain rights in this invention.

### INTRODUCTION

Respiratory conditions afflict millions of people. For example, chronic obstructive pulmonary disease (COPD) is a respiratory condition that afflicts millions of people in the U.S. alone. The disease may be divided into two subgroups, namely chronic bronchitis and emphysema. Chronic bronchitis is characterized by mucus hypersecretion from the conducting airways, inflammation and eventual scarring of the bronchi (airway tubes). Emphysema is characterized by destructive changes and enlargement of the alveoli (air sacs) within the lungs. Many persons with COPD have a component of both of these conditions. COPD patients have difficulty breathing because they develop smaller, inflamed air passageways and have partially destroyed alveoli.

COPD is characterized by peribronchial and perivascular inflammation and largely irreversible airflow obstruction. Long term cigarette smoking (CS) is the major cause of COPD and while the severity of the disease may vary, all long term smokers will develop some degree of lung function impairment. In addition to CS exposure, acute disease exacerbations due to infection contribute significantly to COPD progression. COPD exacerbations are characterized by a worsening of the patient's condition including changes in symptoms such as dyspnea, cough, and sputum production. These exacerbations are typically marked by a visit to healthcare providers often resulting in long hospital stays at the cost of billions of dollars a year in direct costs. Currently, there is no known cure for COPD.

Asthma is another respiratory condition that afflicts millions of people in the U.S., in which a subject's airways are chronically inflamed. Asthma is characterized by infiltration of leukocyte subsets including eosionophils, macrophages and lymphocytes including natural killer (NK) cells that contribute to the sustained inflammation. The disease is characterized by sustained TH2 immune responses including elevated IgE antibody levels that contribute to disease severity. Currently, there is no known cure for asthma.

### SUMMARY

Methods and compositions for the treatment of respiratory conditions, such as asthma and COPD, are provided. Aspects of the subject methods include administering to the subject a composition comprising an inhibitor of NKG2D-mediated activation of leukocytes. Also provided are compositions suitable for use in the subject methods, as well as pharmaceutical preparations thereof.

Methods of the present disclosure include treating or preventing a respiratory condition in a subject, the methods involving administering to the subject a composition including an inhibitor of NKG2D-mediated activation of leukocytes; and a pharmaceutically acceptable vehicle, wherein the composition is administered in an amount effective to reduce or prevent symptoms of the respiratory condition in the subject. Respiratory conditions of interest include, but are not limited to, asthma (e.g., steroid-resistant asthma), and COPD (e.g., chronic bronchitis and/or emphysema). The subject being treated may be one that has been diagnosed with a respiratory condition. Subjects suitable for treatment via methods disclosed herein include mammals, e.g., humans.

Methods of the present disclosure also include methods of treating or preventing a respiratory condition in a subject suffering from or at a significant risk of developing the respiratory condition, the methods involving administering to the subject a composition including an inhibitor of NKG2D-mediated activation of leukocytes; and a pharmaceutically acceptable vehicle, wherein the composition is administered in an amount effective to reduce or prevent symptoms of the respiratory condition in the subject.

In practicing the subject methods, in certain aspects the inhibitor of NKG2D-mediated activation of leukocytes is an antibody, or fragment thereof, that binds to NKG2D and/or an NKG2D ligand. The antibody or fragment thereof may be a monoclonal antibody. In certain aspects, the antibody is a human antibody, a humanized antibody, or a chimeric antibody. Ligands of interest to which an antibody, or fragment thereof, may bind include, but are not limited to, MICA, MICB, ULBP1, ULBP2, ULBP3, ULBP4, ULBP5, and ULBP6. An antibody, or fragment thereof, that binds to an NKG2D ligand may block the ligand from binding to NKG2D and/or prevent NKG2D dependent activation of immune cells.

Where desired, the methods disclosed herein may further include administering an effective amount of a second respiratory condition treatment active agent to the subject, where agents of interest include, but are not limited to, bronchodilators, inhaled corticosteroids, leukotriene modifiers, long-acting beta agonists, combination inhalers, theophylline; immunomodulators, short-acting beta agonists, intravenous corticosteroids, phosphodiesterase-4 (PDE4) inhibitors, and expectorants.

Aspects of the invention also include pharmaceutical compositions comprising a NKG2D inhibitor (e.g., as described above) where the compositions may be configured for use in methods such as those summarized above. Pharmaceutical compositions may contain a first respiratory condition treatment agent comprising an NKG2D inhibitor, and a second respiratory condition treatment active agent (e.g., such as those described above). Also provided are kits comprising an effective amount of a NKG2D inhibitor as an active agent, and instructions for using the composition to treat a respiratory condition in a subject in need thereof.

### BRIEF DESCRIPTION OF THE DRAWINGS

The invention may be best understood from the following detailed description when read in conjunction with the accompanying drawings. Included in the drawings are the following figures:

FIG. 1, Panels A-B provide graphs showing the results of a long term model of allergic airway inflammation induced by exposure to ovalbumin antigen. NKG2D deficient mice, and mice treated with NKG2D antibody, exhibit reduced infiltration of immune cells in bronchoalveolar lavage fluid (BALF).

FIG. 2 provides graphs showing the results of a short-term model of asthma induced by exposure to Aspergillus. NKG2D deficient mice exhibit reduced infiltration of key immune cells in bronchoalveolar lavage fluid (BALF).

FIG. 3, Panels A-B provides graphs showing the results of a long-term Aspergillus model of allergic airway inflammation. NKG2D deficient mice exhibit reduced infiltration of key immune cells in bronchoalveolar lavage fluid (BALF) (Panel A) and in the lung tissue (Panel B).

FIG. 4 provides a graph showing the results of an experiment in which serum from wild type or NKG2D KO mice that had been exposed to the long Aspergillus protocol was analyzed for IgE. Serum from NKG2D KO mice had significantly less IgE compared to that of WT mice.

FIG. 5, Panels A-F show characterization of NK cell markers in a mouse model of COPD. NK cells were isolated from C57BL/6 mice exposed to FA or CS for 6 months and analyzed by flow cytometry. Panels A-B: Total NK cells enumerated by NKp46+ expression. Panels C-D: The percentage of NKp46+ cells expressing the indicated NK cell markers. Panels E-F: The geometric mean fluorescent intensity (MFI) of NK cell receptors. Values are presented as means±SEM. n=5-6 per group. All data representative of two independent experiments.

FIG. 6, Panels A-B show NK cell cytotoxicity against MHC class I-deficient and NKG2D ligand expressing targets in mouse model of COPD. Panel A: Representation of flow cytometry analysis comparing in vivo clearance. CFSE labeled C57BL/6 (CFSE low) cells and C57BL/6 B2m^{-/-} (CFSE high) cells were injected i.v. into receipt mice exposed to exposed to FA or CS. Mice were bled 16 hrs following injections and labeled target cells were analyzed by flow cytometry. Panel B: Time course of NK cell cytotoxicity against B2m^{-/-} target cells, as described in Panel A, in mice exposed to FA or CS for 3-10 months. Data representative of results from four independent experiments.

FIG. 7 shows NK cells of CS exposed mice demonstrate increased cytotoxic activity towards RAE1 expressing cells. NK cells were purified from pooled splenocytes of mice exposed to FA or CS for 6 months and cytotoxicity towards RAE1ε-targets was assessed ex vivo. NK cells and CFSE-labeled RMA cells transfected to express RAE1ε (or mock transfected) were combined at increasing effector to target (E:T) ratios. Cells were incubated for 4 h, harvested and analyzed by flow cytometry as described in the methods. Data is representative of four independent experiments each using NK cells pooled from two mice. Significant differences between groups at all E:T ratios are indicated.

FIG. 8 shows that Klrk1^{-/-} mice do not develop enhanced cellular responses in a mouse model of COPD. NK cells from the pooled spleens of 5 mice were highly purified (>99% NKp46+) and stimulated overnight with cytokines as labeled. IFNγ was quantified by ELISA. The marked (*) axis is the scale representing levels of IFNγ released by IL-12/18 stimulation. Values are presented as means±SEM. Relevant significant differences between groups are highlighted. Data is representative of three independent experiments.

FIG. 9, Panels A-I show CS-exposed Klrk1^{-/-} mice lack enhanced cellular responses associated with influenza infection. Mice were infected with 2×10³ pfu influenza virus and endpoints were measured 4 days after infection. Panels A-B: H&E stained lung sections representing changes in lung inflammation and airways obstruction between groups. Panels C-D: Clara cell secretory protein (CCSP) immunohistochemistry staining of lung sections showing epithelial damage of large airways and small airways. Panels E-F: Total RNA was isolated from lung homogenates of FA- or CS-exposed Klrk1^{+/+} and Klrk1^{-/1} mice with and without influenza infection. Raet1 and Mult1 transcripts were assayed by quantitative RT-PCR and normalized to Rpl32. Panel G: H&E stained lung sections of influenza-infected CS-exposed Klrk1^{-/-} mice which received NK cells from FA or CS-exposed Klrk1^{+/+} mice. Panel H: Semi-quantitative assessment of inflammation severity and distribution. Panel I: Quantitation of the percentage of intrapulmonary airways exhibiting any degree of airways obstruction. (n=4-8 mice per group).

FIG. 10 shows long-term CS exposure does not affect viral clearance in Klrk1^{+/+} or Klrk1^{-/-} mice. Influenza titers in individual mice were determined by plaque assay as described in Methods. Data are presented as plaque-forming units (PFU) per gram of wet lung tissue. All data representative of two independent experiments (n=4-8 mice per group).

### DETAILED DESCRIPTION

Methods and compositions for the treatment of respiratory conditions, such as asthma and COPD, are provided. Aspects of the subject methods include administering to the subject a composition comprising an inhibitor of NKG2D-mediated activation of leukocytes. Also provided are compositions suitable for use in the subject methods, as well as pharmaceutical preparations thereof.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The present disclosure encompasses methods and compositions effective for treating or preventing a respiratory condition in a subject. In certain aspects, the respiratory condition is asthma or COPD. The methods are carried out by administering to the subject a composition comprising an inhibitor of NKG2D-mediated activation of leukocytes, wherein the composition is administered in an amount effective to reduce or prevent symptoms of the respiratory condition in the subject. NKG2D activation may be inhibited by one or more of: (1) depleting the cell surface of NKG2D molecules pre-existing on the cell surface; (2) interfering with the functional interaction between NKG2D and one or more of its ligands expressed in lung tissue or associated secondary lymphoid tissue or otherwise blocking the signaling function of NKG2D; and (3) preventing NKG2D molecules from reaching the cell surface, including interfering with the production of NKG2D at a transcriptional, translational, or post-translation level. In some embodiments, the invention encompasses reducing pre-existing cell surface NKG2D molecules by stimulating their internalization without concurrently causing significant activation that would trigger the effector functions of NKG2D-bearing leukocytes.

The terms “NKG2D,” “NKG2-D,” “KLRK1,” “Klrk1” and “killer cell lectin-like receptor subfamily K, member 1,” as used herein refer to a human killer cell activating receptor gene, cDNA (e.g., Homo sapiens: GENBANK Accession No. NM_{—}007360.3), and/or its gene product (GENBANK Accession No. CAA04925.1), as well as its mammalian counterparts, including wild type and mutant products. Mammalian counterparts of NKG2D include but are not limited to mouse NKG2D (e.g., Mus musculus: GENBANK Accession No. NM_{—}033078.3), rat NKG2D (e.g., Rattus norvegicus: GENBANK Accession No. NM_{—}133512.1), pig NKG2D (e.g., Sus scrofa: GENBANK Accession No. AF285448.1), monkey NKG2D (e.g., Macaca mulatta: GENBANK Accession No. AJ554302.1), and orangutan NKG2D (e.g., Pongo pygmaeus: GENBANK Accession No. AF470403.1). NKG2D inhibitors such as NKG2D antagonists and partial antagonists find use in connection with the disclosed methods and compositions.

Unless otherwise stated, the disclosed methods can be practiced in the context of treating (e.g., reducing the symptoms associated with and/or underlying conditions that are considered causative for a condition either in terms of time such symptoms/conditions exist, spread of such conditions/symptoms, severity of such conditions/symptoms, etc.) or preventing (e.g., reducing the likelihood of developing, delaying the onset of, delaying the severity of post-onset, reducing the severity of upon onset, etc.) an inflammatory disease of the airways or a respiratory condition. Such diseases and/or conditions of interest include, but are not limited to, asthma, steroid-resistant asthma, and COPD (e.g., chronic bronchitis and/or emphysema).

In some instances, the severity of one or more symptoms is reduced by 2 fold or more, such as 5 fold or more, including 10 fold or more. Evaluation of the severity of symptoms may be determined using any convenient protocol, such as by spirometric measure(s) of pulmonary function (e.g., FEV₁, FVC, and their ratio) such as is described in Enright, et al. Am Rev Repir Dis 1991; 143:1215-1233 and Enright, et al. Am J Repir Crit Care Med 1994; 149:S9-S18; inspiratory capacity such as is described in O'Donnell D E. Chest 2000; 117:42 S-47S; dyspnea on effort, wheeze, cough, and sputum production, as measured by, for example the Borg scale (such as is described in Burdon, et al. Am Rev Repir Dis 1982; 126:825-828) or the Medical Research Council dyspnea scale (such as is described in Fletcher, C M. BMJ 1960; 2:1665); exercise tests such as the 6-minute walk test (6-MWT) such as is described in Butland, et al. BMJ 1982; 284:1607-1608 and Solway, et al. Chest 2001; 119:256-270 or the Incremental Shuttle Walk Test such as is described in Singh, et al. Thorax 1992; 47:1019-1024; QOL indexes such as the Saint George's Respiratory Questionnaire described in Jones, et al. Am Rev Respir Dis 1992; 145:1321-1327 or the Chronic Respiratory Questionnaire described in Guyatt, et al. Thorax 1987; 42:773-778; computed tomography scans such as are described in Spouge, et al. J Comput Assist Tomogr 1993; 17:710-713 and Genovois, et al. Am J Respir Crit Care Med 1995; 152:653-657; markers of airway inflammation such as is described in Gizychi, et al. Thorax 2002; 57:799-803; composite outcomes such as are described in Gross, N J Proc Am Thorac Soc 2005; 2:267-271, the Method of Expert Panel Report 2 described in National Asthma Education and Prevention Program, Expert Panel Report 2. Washington, D.C.: Dept of Health and Human Services; 1997. NIH Publication No. 97-4051; National Asthma Campaign in Australia described in Asthma Management Handbook 1998, National Asthma Campaign. South Melbourne, Australia: National Asthma Council Austrailia Ltd, CAN 058 044 634; 1995; and British Guideline on the Management of Asthma Thorax 2003; 58(S1):i1-i94; the dose of inhaled corticosteroids needed to provide control of symptoms such as is described in Colice, G L Clin Mecidine & Research 2004; 2:155-163; changes in sputum eosinophil counts such as is described in Jatakanon, et al. Am Respir Crit Care Med 2000; 161:64-72; exhaled levels of nitric oxide such as is described in Turktas, et al. J Asthma 2003; 40:425-430; and/or bronchial hyperresponsiveness such as is described in Van Den Berge, et al. Am J Respir Crit Care Med 2001; 163:1546-1550; the disclosures of which are each incorporated herein by reference in their entirety.

### NKG2D-Inhibiting Agents

Unless otherwise stated or clearly implied by context, in practicing the disclosed methods, any agent that reduces NKG2D-mediated leukocyte activation in lung tissue or associated secondary lymphoid tissue may be used. Non-limiting examples of such agents include: an NKG2D ligand, or an NKG2D-binding fragment, variant, or derivative thereof; an antibody, or a fragment, variant, or derivative thereof (such as, e.g., an NKG2D-binding antibody); a nucleic acid (or variant or derivative thereof), or a small molecule, that inhibits NKG2D or DAP10 production in a cell; peptides or small molecules that interfere with the formation or function of the NKG2D-DAP10 complex; small molecules that alter NKG2D signal transduction, and combinations of any of the foregoing. Exemplary NKG2D ligands can be found in, for instance, U.S. Pat. No. 6,653,447; Carayannopoulos et al., J Immunol, 169(8):4079-83, 2002; Carayannopoulos et al., Eur J Immunol, 32(3):597-605, 2002; Sutherland et al., J Immunol, 168(2):671-9, 2002; Sutherland et al., Immunol Rev, 181:185-92, 2001; and Cosman et al., Immunity, 14(2):123-33, 2001). Additional NKG2D-inhibiting agents are described, for example, in PCT Publication No. WO2011022334, the disclosure of which is incorporated by reference herein.

The present disclosure encompasses agents that contact NKG2D-expressing cells and reduce the activation of NKG2D-bearing cells when they are subsequently exposed to NKG2D-ligand bearing cells or recombinant NKG2D ligands. Any indicator of this activation may be monitored, including, without limitation, stimulation of DAP10 phosphorylation, stimulation of p85 PI3 kinase, activation of Akt, NKG2D-dependent production of interferon-gamma (IFN-γ) or other cytokines or chemokines, NKG2D-dependent killing of NKG2D-ligand bearing target cells, and the like. One means of assessing the level of NKG2D activation is by measuring the human NK cell killing of NKG2D ligand-bearing target cells. In some embodiments of the present disclosure, useful NKG2D-inhibiting agents are those that cause at least about 20% reduction of NKG2D ligand-induced NKG2D activation in a model system such as that described in US Patent Application Publication No. 2012/0064070, the disclosure of which is incorporated by reference herein; in other embodiments, the agent results in at least about 30%, 40%, 50%, 60%, 70%, 80%, 90%, or more reduction in ligand-induced NKG2D activation. For example, NKG2D ligand-induced activation can be reduced by at least about 30% in the presence of the agent as compared to a control. The control may be, for example, NKG2D-activation in the absence of the agent but under substantially identical conditions in either (a) an individual, (b) a population of substantially similar organisms, using an average value as control, or (c) both. Another means of assessing the level of NKG2D activation is by measuring IFN-γ production in the presence or absence of an NKG2D ligand such as MICA or ULBP. Any method for measuring IFN-γ production may be used, including, without limitation, immunoassays or other assays that measure IFN-γ protein; bioassays that measure IFN-γ activity, and the like. In some embodiments of the present disclosure, useful NKG2D-inhibiting agents are those that cause at least about 20% reduction of NKG2D-mediated IFN-γ production; in other embodiments, the agent results in at least about 30%, 40%, 50%, 60%, 70%, 80%, 90%, or more reduction in NKG2D-mediated IFN-γ production.

In one series of embodiments, the NKG2D-inhibiting agents according to the present disclosure stimulate cellular internalization of NKG2D. Internalization may be assessed by any appropriate means, such as, e.g., by flow cytometry; immunofluorescence microscopy (including, monitoring internalization of an antibody by confocal microscopy); binding assays that detect cell-surface NKG2D, and the like. In some embodiments of the present disclosure, useful NKG2D-inhibiting agents are those that cause at least about 10% reduction in the cell-surface level of NKG2D or a 10% increase in the rate of disappearance of NKG2D from the cell surface, as compared to control when tested in a model system such as that described in US Patent Application Publication No. 2012/0064070, the disclosure of which is incorporated by reference herein; in other embodiments, the agent results in at least about 20%, 30%, 40%, 50%, 60%, 70%, 80%, 90%, 95%, or >99% reduction in the cell-surface level or at least about 20%, 30%, 40%, 50%, 60%, 70%, 80%, 90%, 95% increase in the rate of disappearance of NKG2D.

In some instances, the NKG2D-inhibiting agents according to the present disclosure do not result in significant cytolysis or depletion of NKG2D-expressing cells, including, e.g., one or more of CD8+ T cells, CD4+ T cells, yδ-TcR+ T cells, NKT cells, and CD56/16+ NK cells. The ability of an agent to kill NKG2D-expressing cells may be assessed using any appropriate means, such as, e.g., by detection of dead cells by flow cytometry or microscopy using annexin V or propidium iodide staining, incorporation of Trypan blue, europium assay or chromium release assay. In some embodiments of the invention, useful NKG2D-inhibiting agents are those that exhibit a detectable therapeutic benefit under conditions that preserve the viability at least about 90% of NKG2D-expressing cells. In other embodiments, the agent causes less than about 5%, 10%, 20% 30%, 40%, 50%, 60%, 70%, or 80% reduction in the number of NKG2D-expressing cells.

The present disclosure relates to the inability of natural soluble ligands of NKG2D (such as, e.g., MICA or ULBP) to stimulate internalization of NKG2D in patients suffering from chronic inflammation in a manner similar to internalization that might occur in individuals not suffering from chronic inflammation; without wishing to be bound by theory, it is believed that this phenomenon results at least in part from the high levels of cytokines that accompany chronic inflammatory states. (This phenomenon may be documented by comparing the NKG2D levels on T cells or NK cells in patients suffering from chronic inflammation and in healthy patients; similar NKG2D levels in the two groups, notwithstanding the fact that chronic inflammation is accompanied by high circulating levels of NKG2D ligands, reflect a defect in NKG2D internalization). The present disclosure encompasses agents that stimulate the internalization of NKG2D under conditions in which the natural soluble NKG2D ligands would not be effective or would be less effective in doing so, as well as the use of such agents in the various methods provided herein. Any suitable model system for examining this effect may be used to demonstrate that particular agents possess or exhibit such characteristics, for instance by comparing the effect on NKG2D internalization of a natural soluble ligand and a an inhibiting agent according to the present disclosure, under conditions in which NKG2D-expressing cells are exposed to cytokines (including, without limitation, interleukin-2, interleukin-15, tumor necrosis factor, or combinations of the foregoing) under conditions known to counteract the effect of the natural soluble ligands on internalization. In some embodiments, the NKG2D-inhibiting agents of the invention can cause a reduction in surface NKG2D levels that is at least 10% greater than the reduction in surface NKG2D levels caused by a natural soluble NKG2D ligand, when internalization is measured under conditions (such as, e.g., in the presence of one or more cytokines) that interfere with the ability of the natural soluble ligand to mediate internalization. In other embodiments, the NKG2D-inhibiting agents are at least about 20%, 30%, 40%, 50%, 60%, 70%, 80%, 90%, 95%, or >99% more effective than a natural soluble NKG2D ligand in mediating NKG2D internalization.

NKG2D Ligands

One type of NKG2D-inhibiting agent according to the invention encompasses NKG2D ligands. Typically, such ligands exhibit some modification relative to the natural soluble NKG2D ligands (such as, e.g., soluble forms of MICA, MICB, and ULBP) that renders them effective in stimulating NKG2D internalization under conditions in which the natural soluble ligands are ineffective. For example, soluble forms of MICA and MICB proteins (i.e., lacking the transmembrane and cytoplasmic domains, see, e.g., U.S. Patent Application US 2003/0165835, herein incorporated by reference), or fragments therefrom that retain NKG2D-binding activity, may be chemically cross-linked using conventional methods to form multimeric NKG2D ligands that are capable of binding to more than one NKG2D molecule and thereby stimulating internalization. NKG2D-binding activity may be assessed using any means, including, e.g., competitive binding, flow cytometry, and the like. In another series of embodiments, multimeric NKG2D ligands may be produced by expression of nucleic acids encoding polypeptides having tandem repeats (separated by appropriate spacers) of NKG2D-binding domains derived from MICA, MICB, or ULBP. In another series of embodiments, the ligands may incorporate additional chemical groups, such as, e.g., polyethylene glycol (PEG).

In some embodiments, a NKG2D-inhibiting agent is an agent which interferes with one or more NKG2D ligand binding interactions, e.g., as an inhibitor of NKG2D ligand activity or expression. For example, a suitable inhibitor may be an inhibitor of MICA, MICB, or ULBP expression or activity. In some embodiments, the inhibitor of NKG2D ligand activity or expression is a MICA/B inhibitor or a ULBP inhibitor. In some embodiments, the MICA/B or ULBP inhibitor is MICA/B-specific siRNA or a ULBP-specific siRNA.

Antibodies

The present disclosure encompasses the use of any antibodies that can be used to decrease NKG2D-mediated activation of leukocytes in lung tissue or associated secondary lymphoid tissue, such as, e.g., those that stimulate internalization of NKG2D without significant activation via NKG2D-mediated signaling pathways. Non-limiting examples of such antibodies include antibodies directed against any suitable extracellular or intramembrane epitope of NKG2D; antibodies directed against any suitable extracellular or intramembrane epitope of DAP10; and antibodies directed against a soluble NKG2D ligand or an NKG2D-NKG2D ligand complex. Also encompassed are bispecific antibodies, i.e., antibodies in which each of the two binding domains recognizes a different binding epitope. The amino acid sequence of NKG2D is disclosed, e.g., in U.S. Pat. No. 6,262,244, the amino acid sequence of DAP10 is disclosed in Wu et al., Science 285:730, 1999, and the amino acid sequences of MICA and MICB polypeptides are disclosed, e.g., in U.S. Patent Application US 2003/0165835, all herein incorporated by reference in their entirety. NKG2D ligands against which an antibody, or fragment thereof, may be directed include, but are not limited to, MICA, MICB, ULBP1, ULBP2, ULBP3, ULBP4, ULBP5, ULBP6, and those described in Champsaur and Lanier, Immunological Reviews 235:267 (2010); the disclosure of which is incorporated herein by reference in its entirety.

The term “antibodies” includes antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. Also encompassed by the term are Fab′, Fv, F(ab′)₂, and or other antibody fragments that retain specific binding to antigen, and monoclonal antibodies. An antibody may be monovalent or bivalent.

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH₁) of the heavy chain. Fab fragments differ from Fab′ fragments by the addition of a few residues at the carboxyl terminus of the heavy chain CH₁ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “sFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

Antibodies that may be used in connection with the present disclosure thus can encompass monoclonal antibodies, polyclonal antibodies, bispecific antibodies, Fab antibody fragments, F(ab)₂ antibody fragments, Fv antibody fragments (e.g., V_{H} or V_{L}), single chain Fv antibody fragments and dsFv antibody fragments. Furthermore, the antibody molecules may be fully human antibodies, humanized antibodies, or chimeric antibodies. In some embodiments, the antibody molecules are monoclonal, fully human antibodies.

The antibodies that may be used in connection with the present disclosure can include any antibody variable region, mature or unprocessed linked to any immunoglobulin constant region. If a light chain variable region is linked to a constant region, it can be a kappa chain constant region. If a heavy chain variable region is linked to a constant region, it can be a human gamma 1, gamma 2, gamma 3 or gamma 4 constant region, more preferably, gamma 1, gamma 2 or gamma 4 and even more preferably gamma 1 or gamma 4.

In some embodiments, fully human monoclonal antibodies directed against, e.g., NKG2D or DAP10 are generated using transgenic mice carrying parts of the human immune system rather than the mouse system.

Minor variations in the amino acid sequences of antibodies or immunoglobulin molecules are encompassed by the present invention, providing that the variations in the amino acid sequence maintain at least 75%, e.g., at least 80%, 90%, 95%, or 99% of the sequence. In particular, conservative amino acid replacements are contemplated. Conservative replacements are those that take place within a family of amino acids that are related in their side chains. Whether an amino acid change results in a functional peptide can readily be determined by assaying the specific activity of the polypeptide derivative. Fragments (or analogs) of antibodies or immunoglobulin molecules, can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Sequence motifs and structural conformations may be used to define structural and functional domains in accordance with the invention.

In general, useful anti-NKG2D antibodies according to the present disclosure exhibit an affinity (Kd) for human NKG2D that is at least equal to that of soluble NKG2D ligands. As used herein, the term “affinity” refers to the equilibrium constant for the reversible binding of two agents; “affinity” can be expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. The term “binding” refers to a direct association between two molecules, due to, for example, covalent, electrostatic, hydrophobic, and ionic and/or hydrogen-bond interactions, including interactions such as salt bridges and water bridges.

In some embodiments, the antibodies bind human NKG2D with nanomolar affinity or picomolar affinity. In some embodiments, the antibodies bind human NKG2D with a Kd of less than about 100 nM, 50 nM, 20 nM, 20 nM, or 1 nM.

In some embodiments, useful antibodies include those that reduce the interaction between human NKG2D and one or more of MICA, MICB, ULBP1, ULBP2, ULBP3, ULBP4, ULBP5 and ULBP6. Such blocking antibodies may be identified using conventional competition assays.

Specific anti-NKG2D antibodies which may be employed include, but are not limited to: MS, 21F2, and others described in US Patent Publication US 2010/0056764; MI-6 and others described in US Patent Publication 2012/0148581; 1D11 and others described in US Patent Publication 2005/0158307; and those described in US Patent Publications 2005/0136050, 2006/0280755, 2008/0274047, 2008/0299137, 2010/0272718, 2011/0150870, and 2012/0064070; the disclosures of which are incorporated herein by reference in their entirety.

Expression Modulatory Agents

The present disclosure encompasses inhibition of NKG2D cell surface expression (or expression of a NKG2D ligand) at a transcriptional, translational, or post-translational level. In some embodiments, the inhibitors are nucleic-acid based, including, without limitation, DNA, RNA, chimeric RNA/DNA, protein nucleic acid, and other nucleic acid derivatives.

In some embodiments, the NKG2D inhibitors encompass RNA molecules capable of inhibiting NKG2D production when introduced into an NKG2D-expressing cell (termed RNAi), including short hairpin double-stranded RNA (shRNA). The phrase “RNA interference” and the term “RNAi” refer to the process by which a polynucleotide or double stranded polynucleotide comprising at least one ribonucleotide unit exerts an effect on a biological process through disruption of gene expression. The process includes but is not limited to gene silencing by degrading mRNA, interactions with tRNA, rRNA, hnRNA, cDNA and genomic DNA, as well as methylation of DNA and ancillary proteins.

Non-limiting examples of useful RNAi sequences for inhibiting NKG2D expression include those encoded by the sequences

(SEQ ID NO: 1)
5′-GGATGGGACTAGTACACATTCC-3′;
(SEQ ID NO: 2)
5′-TGGCAGTGGGAAGATGGCTCC-3′;
and
(SEQ ID NO: 3)
5′-CAGAAGGGAGACTGTGCACT CTATGCCTC-3′.

See, e.g., U.S. 2012/0064070, the disclosure of which is incorporated by reference herein. It will be understood that any sequence capable of reducing the cell surface expression of NKG2D (or a NKG2D ligand) may be used in practicing the methods of the present disclosure.

In certain embodiments, the subject RNAi constructs are “short interfering RNAs” or “siRNAs.” These nucleic acids are about 18-30 base pairs in length, such as, e.g., about 21-23 nucleotides in length, corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group. siRNA for use in the present invention can be obtained using a number of techniques known to those of skill in the art. Additionally, the term siRNA and the phrase “short interfering RNA” include nucleic acids that also contain moieties other than ribonucleotide moieties, including, but not limited to, modified nucleotides, modified internucleotide linkages, non-nucleotides, deoxynucleotides and analogs of the aforementioned nucleotides.

### Methods of Treatment

The present disclosure provides methods for preventing and/or treating an inflammatory disease of the airways, e.g., asthma and/or COPD, associated with NKG2D activation of leukocytes. Such syndromes, include, but are not limited to, clinical situations in which induction of stress-related NKG2D ligands (e.g., MICA, MICB, and ULBPs) results in excessive activation and/or expansion of autoreactive T cells and/or NK cells, which may be reflected in increased levels of cytokines such as IL-2, TNF-α, and IL-15.

Accordingly, in a particular aspect, the disclosure provides a method for treating and/or preventing a respiratory condition. The method comprises delivering an effective amount of an agent that reduces ligand-induced NKG2D activation to a patient having a respiratory condition or being identified/diagnosed as being at substantial risk of developing a respiratory condition, such that the respiratory condition is treated or prevented. In certain aspects, the respiratory condition is asthma and/or COPD (e.g., chronic bronchitis and/or emphysema).

In one aspect, the treatment/prevention method is practiced by use of a monoclonal antibody or monoclonal antibody fragment “against” (i.e., that is “specific for” or that “specifically binds to” or that “preferentially binds to”) NKG2D (or an NKG2D ligand). In one aspect, the agent (e.g., an anti-NKG2D mAb or mAb fragment) is an agent that is demonstrated to be effective in ameliorating the respiratory condition in an acceptable model thereof, such as those described herein. In a further aspect, the agent is an antibody that is capable of detectably reducing ligand-induced NKG2D activation of NKG2D-expressing leukocytes without significantly depleting such cells (e.g., causing a reduction of about 10% or less of such cells as compared to a suitable control). In one aspect, the method results in an increase in cellular internalization of NKG2D in a leukocyte comprising surface-exposed NKG2D. In one aspect, the method results in a reduction in the number of leukocytes in the lung tissue of a subject. In one aspect, the method results in a reduction in the number of eosinophils in the lung tissue of a subject. In one aspect, the method results in a reduction in the number of polymorphonuclear cells in the lung tissue of the subject. In one aspect, the method results in a reduction in the number of lymphocytes in the lung tissue of the subject. In one aspect, the method results in a reduction in IgE level in the serum of the subject. The above effects may be assessed by, e.g., comparison with a suitable control.

In practicing the methods of the present disclosure, an NKG2D inhibitor may be administered to a patient as a single dose comprising a single-dose-effective amount for preventing or treating the respiratory condition, or in a staged series of doses, which together comprise an effective amount for preventing or treating the respiratory condition. An effective amount of an NKG2D inhibitor refers to the amount of the inhibitor which, when administered in a single dose or in the aggregate of multiple doses, or as part of any other type of defined treatment regimen, produces a measurable statistical improvement in outcome, as evidenced by at least one clinical parameter associated with the respiratory condition. An effective amount of an NKG2D inhibitor may slow the progression of a disease when compared with patients not receiving the NKG2D inhibitor.

It will be understood that the effective amount of the NKG2D inhibitor, as well as the overall dosage regimen, may vary according to the specifics of the disease and the patient's clinical status, which, in turn, may be reflected in one or more clinical parameters such as clinically accepted disease scores. For example, for asthma, the severity of disease and/or outcome of treatment, may be evaluated by monitoring, e.g., the frequency of bronchospasm and/or peak expiratory flow rate. In general, detectable effects on treatment outcome using the methods and compositions of the present invention include a decrease in the necessity for other treatments (including, e.g., a decrease in the amount and/or duration of other drugs or treatments), a decrease in number and/or duration of hospital stays, a decrease in lost work days due to illness, and the like. It will be further understood that the effective amount may be determined by those of ordinary skill in the art by routine experimentation, by constructing a matrix of values and testing different points in the matrix.

The present disclosure encompasses combined administration of one or more additional agents in concert with an NKG2D inhibitor. It will be understood that, in embodiments comprising administration of combinations of an NKG2D inhibitor with other agents, the dosage of the NKG2D inhibitor may on its own comprise an effective amount and additional agent(s) may further augment the therapeutic benefit to the patient. Alternatively, the combination of the NKG2D inhibitor and the second agent may together comprise an effective amount for preventing or treating the syndrome. It will also be understood that effective amounts may be defined in the context of particular treatment regimens, including, e.g., timing and number of administrations, modes of administrations, formulations, etc.

In some embodiments, the additional agent encompasses one or more long-term asthma control medications, e.g.: a bronchodilator, e.g., theophylline; an inhaled corticosteroid, e.g., fluticasone, budesonide, mometasone, flunisolide, or beclomethasone; a leukotriene modifier, e.g., montelukast, zafirlukast or zileuton; a long-acting beta agonist, e.g., salmeterol or formoterol; a combination inhaler, e.g., fluticasone and salmeterol or budesonide and formoterol; cromolyn sodum; theophylline; an immunomodulator, e.g., omalizumab or mepolizumab.

In some embodiments, the additional agent encompasses one or more quick relief medications, e.g.: a short-acting beta agonist, e.g., albuterol, levalbuterol or pirbuterol; ipratropium; oral and intravenous corticosteroids, e.g., prednisone and methylprednisolone.

In some embodiments, the additional agent encompasses one or more COPD medications, e.g.: a short-acting bronchodilator, e.g. anticholinergics such as ipratropium; a beta agonist, e.g., albuterol or levalbuterol; a combination of bronchodilators (e.g., a combination of albuterol and ipratropium; a long-acting bronchodilator, e.g., an anticholinergic such as tiotropium, or a beta agonist (e.g., salmeterol, formoterol, or arformoterol); a phosphodiesterase-4 (PDE4) inhibitor; a corticosteroid (e.g., prednisone); an expectorant, e.g., guaifenesin; a methylxanthine.

### Pharmaceutical Formulations and Modes of Administration

The present disclosure encompasses pharmaceutical formulations comprising NKG2D inhibitors, which may also comprise one or more pharmaceutically acceptable carriers. Pharmaceutically acceptable carriers include any and all suitable solvents, dispersion media, coatings, antibacterial and antifungal agents, isotonic and absorption delaying agents, and the like that are physiologically compatible with an NKG2D inhibitor or related composition or combination provided by the invention. Examples of pharmaceutically acceptable carriers include one or more of water, saline, phosphate buffered saline, dextrose, glycerol, ethanol and the like, as well as combinations thereof. In many cases, it can be desirable to include isotonic agents, for example, sugars, polyalcohols such as mannitol or sorbitol, or sodium chloride in such a composition. Pharmaceutically acceptable substances also minor amounts of auxiliary substances such as wetting agents or emulsifying agents, preservatives or buffers, which desirably can enhance the shelf life or effectiveness of the NKG2D inhibitor, related composition, or combination. Suitability for carriers and other components of pharmaceutical compositions is determined based on their biocompatibility and the lack of significant negative impact on the desired biological properties of the NKG2D inhibitor, related composition, or combination.

NKG2D inhibitor combinations of interest include, but are not limited to, pharmaceutical formulations comprising NKG2D inhibitors and one or more additional respiratory treatment active agents, where active agents of interest include, but are not limited to, bronchodilators, inhaled corticosteroids, leukotriene modifiers, long-acting beta agonists, combination inhalers, theophylline, immunomodulators, short-acting beta agonists, intravenous corticosteroids, phosphodiesterase-4 (PDE4) inhibitors, expectorants, and the long-term asthma control medications, quick relief medications, and COPD medications described above.

NKG2D inhibitor compositions, related compositions, and combinations according to the invention may be in a variety of suitable forms. Such forms include, for example, liquid, semi-solid and solid dosage forms, such as liquid solutions (e.g., injectable and infusible solutions), dispersions or suspensions, emulsions, microemulsions, tablets, pills, powders, liposomes, dendrimers and other nanoparticles, microparticles, and suppositories. The optimal form depends on the intended mode of administration, the nature of the composition or combination, and the therapeutic application. Formulations also can include, for example, powders, pastes, ointments, jellies, waxes, oils, lipids, lipid (cationic or anionic) containing vesicles, DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration.

NKG2D inhibitor compositions also include compositions comprising any suitable combination of a NKG2D inhibitor peptide and a suitable salt thereof. Any suitable salt, such as an alkaline earth metal salt in any suitable form (e.g., a buffer salt), can be used in the stabilization of NKG2D inhibitors (preferably the amount of salt is such that oxidation and/or precipitation of the NKG2D inhibitor is avoided). Suitable salts typically include sodium chloride, sodium succinate, sodium sulfate, potassium chloride, magnesium chloride, magnesium sulfate, and calcium chloride. Compositions comprising a base and NKG2D inhibitors also are provided.

A composition for pharmaceutical use also can include diluents, fillers, salts, buffers, detergents (e.g., a nonionic detergent, such as Tween-80), stabilizers (e.g., sugars or protein-free amino acids), preservatives, tissue fixatives, solubilizers, and/or other materials known in the art to be suitable for inclusion in a pharmaceutically composition.

In another aspect, compositions of the present disclosure intended for oral administration, for example, may be formulated with an inert diluent or an edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

NKG2D inhibitor compositions, related compositions, and combination compositions can be administered via any suitable route, such as an oral, mucosal, buccal, intranasal, inhalable, intravenous, subcutaneous, intramuscular, parenteral, or topical route. They may also be administered continuously via a minipump or other suitable device. The antibody or other NKG2D inhibitor generally will be administered for as long as the disease condition is present, provided that the antibody causes the condition to stop worsening or to improve. The antibody or other NKG2D inhibitor will generally be administered as part of a pharmaceutically acceptable composition as described elsewhere herein. The antibody may be administered by any suitable route, but typically is administered parenterally in dosage unit formulations containing conventional pharmaceutically acceptable carriers, adjuvants, and the like (stabilizers, disintegrating agents, anti-oxidants, etc.). The term “parenteral” as used herein includes, subcutaneous, intravenous, intraarterial, intramuscular, intrasternal, intratendinous, intraspinal, intracranial, intrathoracic, infusion techniques and intraperitoneal delivery. Most commonly, an antibody will be administered intravenously or subcutaneously. Routes of injection also include injection into the muscle (intramuscular, IM); injection under the skin (subcutaneous, SC); injection into a vein (intravenous, IV); injection into the abdominal cavity (intraperitoneal, IP); and other delivery into/through the skin (intradermal, ID, usually by multiple injections).

### Subjects Suitable for Treatment Via the Disclosed Methods and Compositions

The present disclosure provides methods and related compositions for treating and/or preventing respiratory conditions, such as asthma and COPD. Suitable subjects for treatment via the disclosed methods and/or compositions are subjects having a respiratory condition or being identified/diagnosed as being at substantial risk of developing a respiratory condition. Such subjects include, e.g., those suffering from acute and/or allergic airway inflammation, those diagnosed with asthma and/or COPD, those identified/diagnosed as being at substantial risk of developing asthma and/or COPD. Suitable subjects may include, e.g., subjects suffering from a condition characterized by induction of stress-related NKG2D ligands (e.g., MICA, MICB, and ULBPs) which results in excessive activation and/or expansion of autoreactive T cells and/or NK cells, and which may be reflected in increased levels of cytokines such as IL-2, TNF-α, and IL-15.

Subjects suitable for treatment via the disclosed methods and/or compositions may include, e.g., subjects exhibiting frequent wheezing during the first 3 years of life, subjects having a parental history of asthma or eczema, subjects exhibiting eosinophilia, subjects exhibiting wheezing without colds, subjects exhibiting allergic rhinitis, subjects who smoke or used to smoke, subjects who have a family history of COPD, and subjects who have long-term exposure to respiratory irritants (e.g., secondhand smoke, air pollution, chemical fumes, dust from the environment or workplace, and the like).

The subject methods and compositions may be applied to a variety of subjects. In many embodiments the subjects are “mammals” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the subjects are humans. The subject methods may be applied to human subjects of both genders and at any stage of development (i.e., neonates, infant, juvenile, adolescent, adult), where in certain embodiments the human subject is a juvenile, adolescent or adult. While the present invention may be applied to a human subject, it is to be understood that the subject methods may also be carried-out on other animal subjects (that is, in “non-human subjects”) such as, but not limited to, birds, mice, rats, dogs, cats, livestock and horses. Accordingly, it is to be understood that any subject in need of treatment for a condition according to the present disclosure is suitable.

### Kits

Another aspect of the present disclosure provides a kit comprising a NKG2D inhibitor, related composition, or combination, pharmaceutically carrier, and optionally other pharmaceutical composition components. A kit may include, in addition to the NKG2D inhibitor, diagnostic or therapeutic agents. A kit may also include instructions for use in a diagnostic or therapeutic method. In one series of embodiments, the kit includes a NKG2D inhibitor, related compound, or combination composition in a highly stable form (such as in a lyophilized form) in combination with pharmaceutically acceptable carrier(s) that can be mixed with the highly stable composition to form an injectable composition.

In one embodiment, the present invention provides a kit comprising: (a) a therapeutically effective amount as described herein of an agent that inhibits NKG2D activation or signaling combined with a pharmaceutically acceptable carrier; and (b) instructions for use. In another embodiment, the present invention provides a kit comprising: (a) a therapeutically effective amount as described herein of an agent that blocks the NKG2D ligand binding interaction combined with a pharmaceutically acceptable carrier; and (b) instructions for use. Instructions for use may be provided in any suitable format known in the art, e.g., as a part the packaging, as a packaging insert, or on a computer readable medium.

Non-limiting exemplary embodiments of the present disclosure are provided as follows:
1. A method of treating or preventing a respiratory condition in a subject, said method comprising:
   administering to the subject a composition comprising an inhibitor of NKG2D-mediated activation of leukocytes; and
   a pharmaceutically acceptable vehicle, wherein the composition is administered in an amount effective to reduce or prevent symptoms of the respiratory condition in the subject.
2. The method of 1, wherein the respiratory condition is asthma.
3. The method of 1, wherein the respiratory condition is COPD.
4. The method of 3, wherein the subject has emphysema.
5. The method of any one of 1-4, wherein the inhibitor is an antibody or fragment thereof that binds to NKG2D.
6. The method of any one of 1-5, wherein the inhibitor is an antibody or fragment thereof that binds to an NKG2D ligand.
7. The method of 6, wherein the NKG2D ligand is selected from MICA, MICB, ULBP1, ULBP2, ULBP3, ULBP4, ULBP5, and ULBP6.
8. The method of any of 5-7, wherein the antibody is a monoclonal antibody.
9. The method of 8, wherein the monoclonal antibody is human antibody, a humanized antibody, or a chimeric antibody.
10. The method of any of 5-9, wherein the antibody or fragment thereof increases cellular internalization of NKG2D in a leukocyte comprising surface-exposed NKG2D.
11. The method of any one of 1-10, wherein the administering results in a reduction in the number of leukocytes in the lung tissue of the subject.
12. The method of any one of 1-10, wherein the administering results in a reduction in the number of eosinophils in the lung tissue of the subject.
13. The method of any one of 1-10, wherein the administering results in a reduction in the number of polymorphonuclear cells in the lung tissue of the subject.
14. The method of any one of 1-10, wherein the administering results in a reduction in the number of lymphocytes cells in the lung tissue of the subject.
15. The method of any one of 1-10, wherein the administering results in a reduction in IgE level in the serum of the subject.
16. The method of any one of 1-15, wherein the inhibitor reduces expression of NKG2D in the subject.
17. The method of any one of 1-16, wherein the subject suffers from or is at a significant risk of developing steroid responsive asthma.
18. The method of any one of 1-17, wherein the subject exhibits one or more of the following: frequent wheezing during the first 3 years of life, parental history of asthma or eczema, eosinophilia, wheezing without colds, and allergic rhinitis.
19. The method of any one of 1-18, further comprising administering to the subject an effective amount of a second active agent, wherein the second active agent is administered in an amount effective to reduce or prevent symptoms of the respiratory condition in the subject.
20. The method of 19, wherein the second active agent is selected from bronchodilators, inhaled corticosteroids, leukotriene modifiers, long-acting beta agonists, combination inhalers, theophylline, immunomodulators, short-acting beta agonists, intravenous corticosteroids, phosphodiesterase-4 (PDE4) inhibitors, and expectorants.
21. A method of treating or preventing a respiratory in a subject suffering from or at a significant risk of developing the respiratory condition, said method comprising:
   administering to the subject a composition comprising an inhibitor of NKG2D-mediated activation of leukocytes; and
   a pharmaceutically acceptable vehicle, wherein the composition is administered in an amount effective to reduce or prevent symptoms of the respiratory condition in the subject.
22. The method of 21, wherein the respiratory condition is asthma.
23. The method of 21, wherein the respiratory condition is COPD.
24. The method of 23, wherein the COPD is emphysema.
25. The method of any one of 21-24, wherein the inhibitor is an antibody or fragment thereof that binds to NKG2D.
26. The method of any one of 21-25, wherein the inhibitor is an antibody or fragment thereof that binds to an NKG2D ligand.
27. The method of 26, wherein the NKG2D ligand is selected from MICA, MICB, ULBP1, ULBP2, ULBP3, ULBP4, ULBP5, and ULBP6.
28. The method of any one of 25-27, wherein the antibody is a monoclonal antibody.
29. The method of 28, wherein the monoclonal antibody is human antibody, a humanized antibody, or a chimeric antibody.
30. The method of any one of 25-29, wherein the antibody or fragment thereof increases cellular internalization of NKG2D in a leukocyte comprising surface-exposed NKG2D.
31. The method of any one of 21-30, wherein the administering results in a reduction in the number of leukocytes in the lung tissue of the subject.
32. The method of any one of 21-30, wherein the administering results in a reduction in the number of eosinophils in the lung tissue of the subject.
33. The method of any one of 21-30, wherein the administering results in a reduction in the number of polymorphonuclear cells in the lung tissue of the subject.
34. The method of any one of 21-30, wherein the administering results in a reduction in the number of lymphocytes cells in the lung tissue of the subject.
35. The method of any one of 21-30, wherein the administering results in a reduction in IgE level in the serum of the subject.
36. The method of any one of 21-30, wherein the inhibitor reduces expression of NKG2D in the subject.
37. The method of any one of 21-30, wherein the subject suffers from or is at a significant risk of developing steroid responsive asthma.
38. The method of any one of 21-37, wherein the subject exhibits one or more of the following: frequent wheezing during the first 3 years of life, parental history of asthma or eczema, eosinophilia, wheezing without colds, and allergic rhinitis.
39. The method of any one of 21-38, further comprising administering to the subject an effective amount of a second active agent, wherein the second active agent is administered in an amount effective to reduce or prevent symptoms of the respiratory condition in the subject.
40. The method of 39, wherein the second active agent is selected from bronchodilators, inhaled corticosteroids, leukotriene modifiers, long-acting beta agonists, combination inhalers, theophylline, immunomodulators, short-acting beta agonists, intravenous corticosteroids, phosphodiesterase-4 (PDE4) inhibitors, and expectorants.
41. A pharmaceutical composition comprising (a) a first respiratory condition treatment comprising an inhibitor of NKG2D-mediated activation of leukocytes as an active agent and (b) a second respiratory condition treatment active agent.
42. The pharmaceutical composition of 41, wherein the second respiratory condition treatment active agent is selected from bronchodilators, inhaled corticosteroids, leukotriene modifiers, long-acting beta agonists, combination inhalers, theophylline, immunomodulators, short-acting beta agonists, intravenous corticosteroids, phosphodiesterase-4 (PDE4) inhibitors, and expectorants.
43. A kit for treating a respiratory condition comprising:
   (a) a composition comprising an effective amount of an inhibitor of NKG2D-mediated activation of leukocytes as an active agent; and
   (b) instructions for using the composition to treat a respiratory condition in a subject in need thereof.
44. The kit according to 43, further comprising an effective amount of a second active agent for treating the respiratory condition.
45. The kit according to 44, wherein the second active agent is selected from bronchodilators, inhaled corticosteroids, leukotriene modifiers, long-acting beta agonists, combination inhalers, theophylline, immunomodulators, short-acting beta agonists, intravenous corticosteroids, phosphodiesterase-4 (PDE4) inhibitors, and expectorants.

### EXAMPLES

As can be appreciated from the disclosure provided above, the present disclosure has a wide variety of applications. Accordingly, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

### Materials and Methods

The following are general materials and protocols used in Examples 6-10 below.

### Mice

C57BL/6J and C57BL/6J B2 m^{-/-} mice (female, 8 to 10 wk old) were purchased from The Jackson Laboratory (Bar Harbor, Me.). NKG2D-deficient (Klrk1^{-/-}) mice were generated as described by Guerra, et al. 2008. Immunity 28:571-580; the disclosure of which is incorporated herein by reference. All of the experimental protocols were reviewed and approved by the Institutional Animal Care and Use Committee at the University of Cincinnati Medical Center.

### Reagents

The following antibodies and immunological reagents were used: NKp46 (29A1.4), NK1.1 (PK136), CD49b (DX5), CD94 (18d3), Streptavidin-APC, NKG2D (CX5), 2B4 (eBio244F4), 7-AAD, CFSE, and IFNγ ELISA (eBioscience); IL-2 and IL-12 (Peprotech); IL-18 (R&D systems); Dynabead CD49b+NK cell isolation kit (Invitrogen); poly(I:C) (HMW) (Invivogen); anti-asialo GM1 (Wako); α-CCSP polyclonal antibody (Seven Hills Bioreagents).

### Mouse Model of COPD

Mice were exposed to either filtered, room air (FA) or cigarette smoke (CS) generated from 3R4F Kentucky Reference Cigarettes (University of Kentucky, Lexington, Ky.) as previously described by Motz, et al. 2008 J Immunol. 181:8036-8043; the disclosure of which is incorporated by reference herein. CS exposures were carried out with a TE-10z smoking machine attached to an exposure chamber (Teague Enterprises, Woodland, Calif.). The concentration of the smoke/air mixture was maintained at 150±15 mg/m³ total suspended particulates. Particulate concentrations were determined by weighing vacuum-drawn total particulate deposition onto filters connected to the chambers. Mice were exposed whole body in the exposure chamber for 4 h/d, 5 d/wk for 6 months.

### Leukocyte Isolation

Mice were euthanized with an i.p. injection of sodium pentobarbital followed by exsanguination. Lungs were perfused with 6 ml 1×PBS containing 0.6 mM EDTA. Lungs and spleens were withdrawn aseptically, and leukocytes were isolated as previously described by Motz, et al. 2008 J Immunol. 181:8036-8043.

### NK Purification and Cell Stimulation

Spleens from five mice were pooled and splenocytes isolated as described above and resuspended in cRPMI (RPMI 1640 with 2.05 mM I-glutamine (HyClone, Waltham, Mass.) containing 10% FBS, 1% sodium pyruvate, 100 U/ml penicillin, 100 μg/ml Streptomycin, and 1× nonessential amino acids (MP Biomedicals, Solon, Ohio). Cell resuspension was followed by a 20 min plastic adherence plating step at 37° C. and 5% CO₂ which greatly reduces the presence of contaminating adherent cells. After plating, remaining leukocytes were enriched for NK cells by positive selection following the manufacturers' protocol for the Dynabead FlowComp Mouse CD49b NK isolation kit (Invitrogen). After enrichment NK cells were >60% pure. The remaining cells were stained with NKp46 and sorted by flow cytometry for NK cells resulting in a purity>99%. A total of 2.0×10⁵ cells in 100 μl of cRPMI containing 20 U/ml mouse rIL-2 (PeproTech, Rocky Hill, N.J.) were aliquoted per well into a 96-well round-bottom culture plate (Costar, Cambridge, Mass.) and cultured at 37° C. and 5% CO₂. Cells were rested 2 h then stimulated for 16 h with IL-18 (10 μg/ml), or IL-12+IL-18 (10 μg/ml). Supernatants were harvested and assayed for IFN-γ production by ELISA (ebioscience).

### In Vivo Cytotoxicity

In vivo cytotoxicity was assessed according to a modified procedure previously described by Barnes, et al. 2010. J. Immuno. 184:3743-3754; the disclosure of which is incorporated by reference herein. Splenocytes were isolated from wild-type C57BL/6J mice or C57BL/6J B2 m^{-/-} mice deficient in MHC Class I molecule expression. Cells were washed twice with PBS+0.2% horse serum (HS) and re-suspended at 10×10⁶ cells/ml. The C57BL/6J B2 m^{-/-} cells were stained with 5 μM CSFM (ebioscience) and the wild-type C57BL/6 cells were stained with 0.5 μM CSFM (ebioscience). Cells were incubated for 8 min at 37° C. and the reaction was stopped by adding ice-cold PBS+20% HS. Cells were washed 2× with PBS+0.2% HS and resuspended at 3×10⁷ cells/ml in PBS. Equal numbers of cells (3.0×10⁶ in 100 μl each) were combined and administered by tail vein injection into FA- or CS-exposed mice. After 16 hrs, ˜200 μl of blood was collected via mandibular bleed using a 4 mm animal lancet (Goldenrod). RBCs were lysed using 1×RBC Lysis solution (Qiagen) then fixed in 1% paraformaldehyde for 20 min at 4° C. Cells were spun at 300×g and the supernatant discarded. Pellets were resuspended in 100 μl of a 2.5% saponin solution. The cells were analyzed by flow cytometry. Cytotoxicity was calculated as follows: (number of CFSE high cells in sample/number of CFSE low cells in sample)/(number of CFSE high cells injected/number of CFSE low cells injected). Flow cytometry was performed using a FACSCalibur (BD Biosciences, San Jose, Calif.). The data were analyzed using the FlowJo software (Tree Star, Ashland, Oreg.).

### Ex Vivo Cytotoxicity

For NK cell enrichment, spleen leukocytes from two mice were isolated, pooled and enriched using Dynabead FlowComp Mouse CD49b NK isolation kit described above (Invitrogen). Cells were resuspended in cRPMI containing 20 U/ml mouse rIL-2 at effector numbers described in the text and added to 5 ml polystyrene round-bottom tubes. Cells were allowed to rest for 2 h at 37° C. and 5% CO₂. RMA-Mock and RMA-Raet1£ cells (kindly provided by Dr. Lewis Lanier, UCSF) were stained with 0.5 μM CFSE (eBioscience) as described above. RMA cells were resuspended in cRPMI and ˜20,000 stained cells were added to tubes containing purified NK cells for a total volume of 400 μl. Combined cells were spun at 300×g and allowed to incubate at 37° C. and 5% CO₂ for 4 h. After incubation, cells were washed in FACS buffer and resuspended in 300 μl buffer. Prior to flow cytometry analysis, 2.5 μl of 7-AAD (eBioscience) was added to each tube. Cytotoxicity was calculated with the following equation using % 7-AAD(−) cells for each group: ((Background-Test)/Background)×100. % Cytotoxicity.

### RT-PCR

Frozen tissue was homogenized using a Tissumizer (Tekmar Co.) and total RNA was isolated with Trizol reagent (Invitrogen). DNase treatment to remove residual DNA was performed using the Turbo DNA-free kit (Ambion). Reverse transcription of total RNA was performed using the high-capacity cDNA Archive kit (Applied Biosystems). FAM labeled probes used for RT-PCR were Ulbp1 (Mult1) (Mm01180648_ml), Raet1 (Mm04206137_gh), and Rpl32 (Mm02528467_g1) (Applied Biosystems). Quantitative reverse transcription-PCR(RT-PCR) was performed using TaqMan universal PCR master mix (Applied Biosystems) on an Applied Biosystems 7300 real-time PCR system. Expression of mRNA was quantified by the ΔΔC_{T} method using Rpl32 as the endogenous control.

### Virus Infection

The influenza A virus HKx31 (H3N2) was used in this study. The virus was passaged in embryonated chicken eggs by standard procedures and titrated on Madin-Darby canine kidney (MDCK) cells. Mice were infected with 2×10³ pfu of virus through noninvasive oral aspiration as described previously by Glasser, et al. 2009. Am J Physiol Lung Cell Mol Physiol 297:L64-72; the disclosure of which is incorporated by reference herein. Infection was allowed to proceed for 4 days at which time the mice were euthanized. Lungs were clamped at the left bronchus and the left lung was frozen for viral titer while the right lung was inflation formalin-fixed and paraffin-embedded as previously described by Borchers, et al. 2006. Infect Immun 74:2578-2586; the disclosure of which is incorporated herein by reference.

### Pathology Assessment and Immunohistochemistry

To assess pulmonary inflammation, paraffin sections of lungs were stained with hematoxylin and eosin. Histological scores for alveolar, peribronchial, and perivascular inflammation was semi-quantitatively scored as the sum of the severity [absent (0), minimal (1), slight (2), moderate (3), strong (4), or severe (5)] and distribution [no inflammation (0) to diffuse (5)] by two analysts blinded to the treatment groups. At least four non-sequential sections were used for each mouse. Airways obstruction was quantitated as the percentage of intrapulmonary airways exhibiting any degree of coalesced luminal debris (i.e., epithelial cells, leukocytes, mucus). Clara cell secretory protein (CCSP) immunohistochemistry was performed on slides to assess airway damage after influenza infection using a polyclonal antibody to mouse CCSP (Seven Hills Bioreagents). The primary antibody was used at 1:20,000 dilution with a goat anti-rabbit biotinylated secondary antibody at 1:200 dilution (Vector Laboratories) after citrate antigen retrieval.

### NK Cell Transfer

For NK cell enrichment, spleen and lung leukocytes from six FA or CS exposed mice were isolated, pooled and enriched using Dynabead FlowComp Mouse CD49b NK isolation kit described above (Invitrogen). Cells were then stained and sorted for CD3⁻ CD49b⁺ populations by flow cytometry. Cells were checked by NKp46 stain to confer >90% NK cells. Cells were washed and resuspended in PBS at a concentration of 2.5×10⁶ cells/ml and 200 μl was administered through tail vein injection into CS Klrk1^{-/-} recipient mice. Mice were allowed to rest for 24 hrs before viral infection as described.

### Plaque Assay

Monolayers of MDCK cells were infected with serial dilutions of lung supernatant after homogenization of frozen lung. The infected cell monolayers were incubated for 1 hr at 37° C. to facilitate viral adsorption and then washed with phosphate-buffered saline. After washing, the MDCK cell monolayers were treated with minimal essential medium containing 1 mg/ml trypsin and 0.8% agarose. The infected monolayers were incubated for 72 hrs at 37° C. At 72 hrs post-infection, the agarose containing medium was gently removed and the monolayers were stained with crystal violet to visualize the influenza virus plaques.

### Statistics

Significant differences among groups were identified by t-test or one-way ANOVA wherever appropriate. Significant differences in histological comparisons where determined using the Mann-Whitney Test. Differences between means were considered significant when the p-value was <0.05.

### Example 1

### NKG2D Contributes to the Pathogenesis of Airway Inflammation

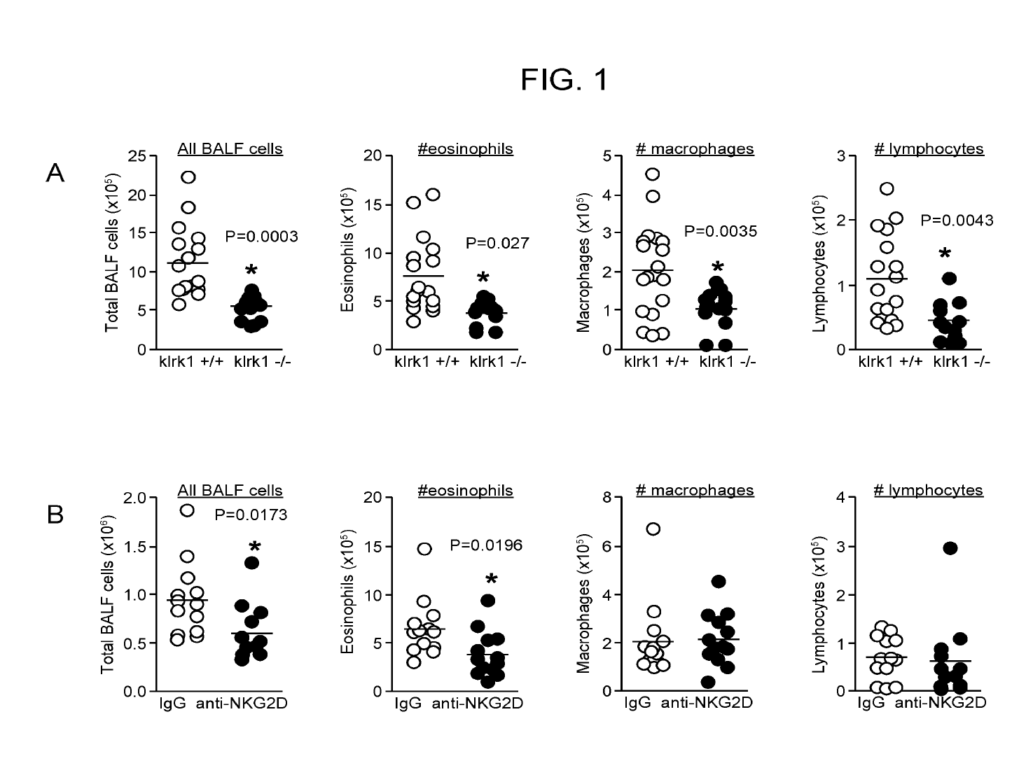

Various mouse models were utilized to test the possibility that NKG2D contributes to the pathogenesis of airway inflammation. The first model tested was a model in which mice are first immunized intraperitoneally with a foreign protein, ovalbumin (mixed with aluminum hydroxide as an adjuvant) to prime ovalbumin specific immune cells. Later, the mice are exposed to aerosolized ovalbumin for several days, which leads to allergic airway inflammation that shares important features with human asthma. Initial comparisons of normal mice called (Klrk1+/+ mice in the FIG. 1) and knockout mice lacking the NKG2D receptor gene (Klrk1−/− mice) were made. The accumulation of immune cells in the bronchoalveolar fluid (BALF) harvested from the lungs of the mice was monitored as a known indication of asthma. As shown in FIG. 1, Panel A, compared to Klrk1+/+ mice, Klrk1−/− mice accumulated fewer leukocytes in the BALF (“All BALF cells”). Analysis of specific cell types showed that there were fewer eosinophils (a key cell type mediating asthma), fewer macrophages and fewer lymphocytes in the knockouts. These data suggested that NKG2D plays an important role in the development of asthma. It was then tested whether injecting normal mice with antibodies that bind NKG2D and block ligand binding would inhibit the disease in normal mice. For these studies, the antibody treatment was restricted to the 4 days at the end of the protocol when the mice were exposed to aerosolized ovalbumin. As shown in FIG. 1, Panel B, treatments with NKG2D antibody significantly inhibited the accumulation of leukocytes in BALF, and also specifically inhibited the accumulation of eosinophils. These data suggest that NKG2D antibodies inhibit the accumulation in BALF of a key cell type that causes allergic airway inflammation, eosinophils.

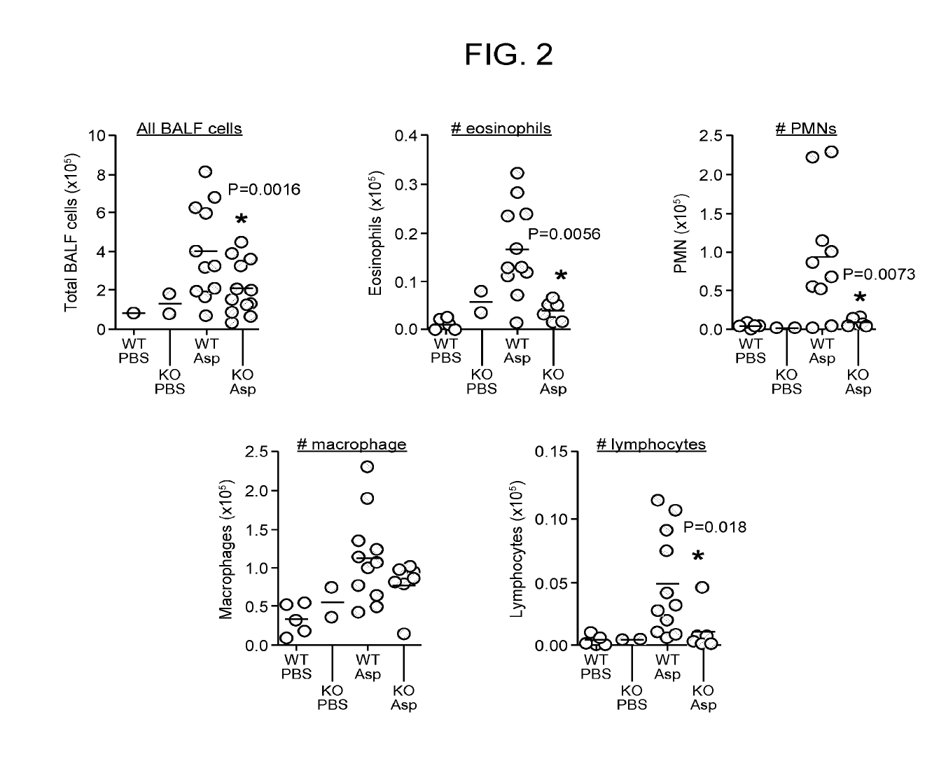

Because the ovalbumin antigen is not a natural antigen involved in asthma the role of NKG2D in two other models was tested, both of which involved exposing mice intranasally to the fungus Aspergillus niger, which may be a natural cause of asthma and can lead to asthma exacerbation. An acute model was tested in which mice are exposed intranasally to a cell wall extract of Aspergillus for 3 days. Shortly thereafter normal mice develop lung inflammation. The rapid onset of symptoms in this model suggests that it is mediated primarily by innate immune cells. As shown in FIG. 2, wildtype mice exposed to Aspergillus accumulate leukocytes in the BALF, including eosinophils, polymorphonuclear cells (PMN), macrophages and lymphocytes. In contrast, NKG2D knockout mice (“KO”) exhibit a significant reduction in leukocytes, including eosinophils, PMNs and lymphocytes. These data suggest that NKG2D also plays a role in the acute model of asthma induced by exposure to Aspergillus and that NKG2D can contribute to acute airway inflammation.

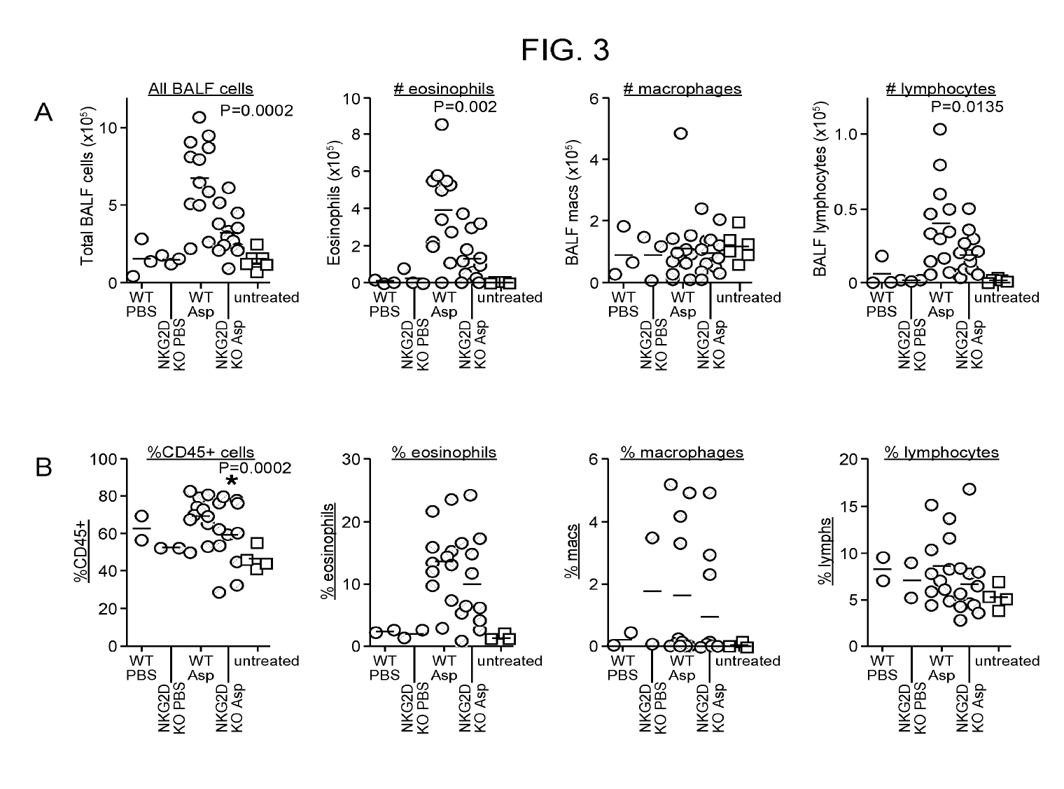

A long-term model resulting from sustained Aspergillus exposure was also tested, which is more characteristic of allergic asthma, including enhanced production of IgE. IgE is known to play an important role in allergic asthma in humans, and agents that prevent IgE production are therapeutic for asthma patients. As shown in FIG. 3, Panel A, wildtype mice exposed intranasally to Aspergillus for 3 weeks showed increased numbers of BALF cells, including eosinophils and lymphocytes, compared to mice exposed to buffered saline solution (PBS). In NKG2D KO mice exposed to Aspergillus, the number of total BALF cells was significantly reduced. Among the BALF cells, eosinophils and lymphocytes were specifically reduced in the knockout mice. The lungs were dissociated to generate a cell suspension, and the content of different cell types was examined. A specific reduction of CD45+ cells (leukocytes) was observed in the lung tissue. The other populations showed variability, possibly due to experimental conditions, so it is not yet clear whether these cell types are reduced in the lung tissue. Notably, eosinophils, macrophages and lymphocytes all trended lower, but the differences were not statistically significant.

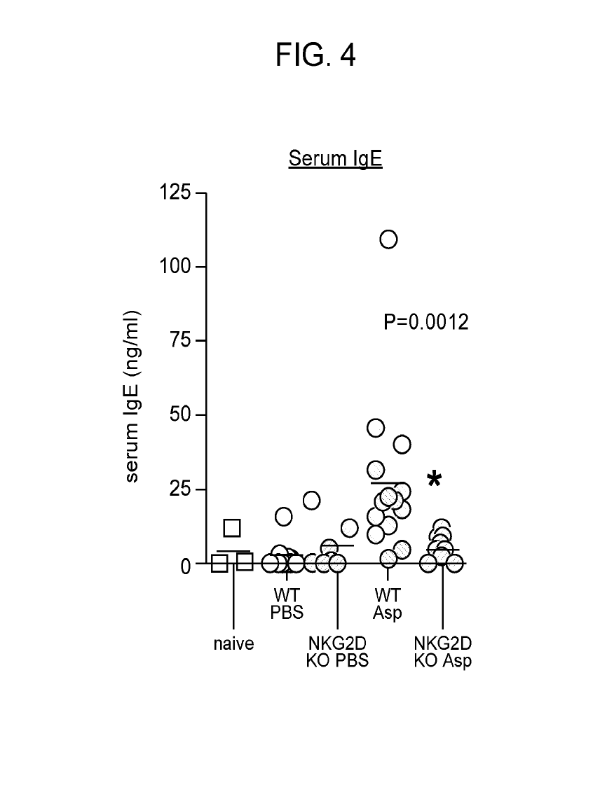

As another key hallmark of allergic asthma, experiments were conducted to determine whether the induction of increased IgE levels is NKG2D dependent in the long-term Aspergillus model. As shown in FIG. 4, IgE levels were sharply increased in WT mice exposed to Aspergillus. However, NKG2D knockout mice showed a substantial and highly significant reduction in IgE levels in the serum. These findings showed that key cell types and antibodies mediating allergic airway inflammation are NKG2D-dependent, and were inhibited (where tested) in mice exposed to NKG2D antibodies. The data as a whole suggest that blocking NKG2D may be an effective approach for treating asthma/airway inflammation in human patients.

Specifics of the above experiments are provided below, in Examples 2-5. All of the experimental protocols used in such Examples were reviewed and approved by the Institutional Animal Care and Use Committee at the University of California, Berkeley.

### Example 2

### Long-Term Model of Allergic Airway Inflammation Induced by Exposure to Ovalbumin

In this model, mice are injected intra-peritoneally with 200 μg of chicken ovalbumin and 1 mg of aluminum hydroxide mixed in 200 μl on day 0 and day 7. On day 14-17, mice are exposed to 6% aerosolized ovalbumin for 25 minutes/day for 4 consecutive days. The mice were euthanized 24 hrs after the last exposure to (day 18).

In FIG. 1, Panel A, wildtype and NKG2D knockout mice were compared. In FIG. 1, Panel B, mice were treated intra-peritoneally on days 14 and 16 (immediately before the first and third exposures to aerosolized ovalbumin) with 100 μg of MI-6, a blocking NKG2D antibody, or isotype control IgG. On day 21, lungs were lavaged with PBS and the fluid (BALF) was collected. Cells were enumerated and analyzed by flow cytometry. The data show the number of BALF cells as well as the number of the indicated types of cells per mouse (2 lungs).

The results show that the NKG2D-deficient mice had significantly fewer total BALF cells, with a significantly lower content of eosinophils, macrophages, and lymphocytes (Panel A). Furthermore, mice treated with an antibody to NKG2D during the “recall stage” only exhibited significantly lower content of BALF cells and eosinophils (Panel B), providing evidence that NKG2D antibodies may be efficacious for treating allergic airway inflammation.

### Example 3

### Short-Term Model of Asthma Induced by Exposure to Aspergillus

In this model, mice were exposed intranasally on day 0 and day 3 to PBS containing cell wall extract of the fungi Aspergillus niger (50 μg in 50 μl), or PBS alone, resulting in acute allergic airway inflammation. The rapid onset of inflammation in this model suggests it is mediated primarily by innate immune cells.

Wildtype and NKG2D knockout mice were compared. At the end of the protocol, lungs were lavaged with PBS and the fluid (BALF) was collected. Cells were enumerated and analyzed by flow cytometry. The data show the number of BALF cells as well as the number of the indicated types of cells per mouse.

The results, depicted in FIG. 2, show that the NKG2D-deficient mice had significantly fewer total BALF cells, with a significantly lower content of eosinophils, PMNs (polymorphonuclear cells) and lymphocytes. The results indicate that NKG2D can contribute to acute allergic inflammatory responses.

### Example 4

### Long-Term Model of Asthma Induced by Exposure to Aspergillus

In this model, mice are exposed intra-nasally 3 times a week for 3 weeks to PBS containing cell wall extract of the fungi Aspergillus niger (50 μg in 50 μl), or PBS alone, to mimic chronic allergen exposure leading to allergic airway inflammation. Both innate and adaptive immune functions contribute to inflammation in this model.

Wildtype and NKG2D knockout mice were compared. At the end of the protocol, lungs were lavaged with PBS and the fluid (BALF) was collected. The lungs were perfused with PBS, digested with collagenase, and the cell suspensions were collected. Cells in both preparations were enumerated and analyzed by flow cytometry. The results are shown in FIG. 3 as follows: Panel A: BALF cells, showing the number of cells/per mouse (2 lungs); and Panel B: Cells in dissociated lung cell suspensions, showing the % CD45+ cells and % of specific cell types, of total cells in the suspensions. The results show that the NKG2D-deficient mice had significantly fewer total BALF cells, with a lower content of eosinophils and lymphocytes (Panel A). Furthermore, the NKG2D-deficient mice had significantly fewer CD45+ cells in the lungs compared to WT mice (Panel B).

### Example 5

### Induction of Increased IgE Levels is NKG2D Dependent in the Long-Term Aspergillus Model

Serum from wild type or NKG2D KO mice that had been exposed to the long aspergillus protocol (above) was analyzed for IgE. Serum from NKG2D KO mice had significantly less IgE compared to that of WT mice. See FIG. 4. These results indicate that NKG2D contributes to elevated levels of serum IgE, a functional hallmark of chronic allergen exposure and asthma.

### Example 6

### Expression of NK Cell Cytotoxicity Receptors is Unchanged in Mouse Model of COPD

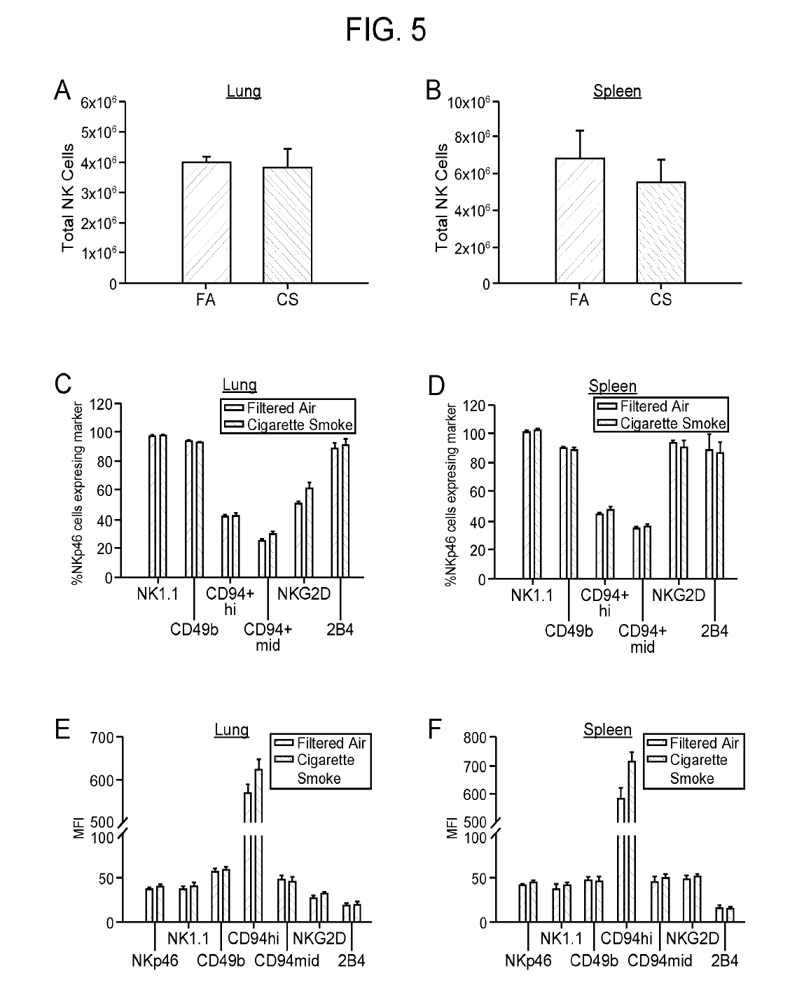

NK cells are the predominate producers of IFNγ in response to viral ligands and this production is enhanced after CS exposure. This enhanced NK cell response is independent of changes in IFN-α, IL-12, or IL-18. The possibility that this altered NK cell phenotype is associated with changes in activating/inhibiting receptor expression on NK cells from mice exposed long-term to CS was explored. The levels of several key receptors were examined, including NKp46, NK1.1, NKG2D, and CD244 (2B4) on NK cells in our mouse model of COPD. The total numbers of NK cells isolated from the lung and spleen was unaltered by long term CS exposure (FIG. 5, Panels A-B). The percentage of NK cells (identified as Nkp46+) expressing the activating receptors NK1.1, NKG2D, and CD244 (2B4), and their receptor density were not different between exposure groups (FIG. 5, Panels C-F). CD94 expression was also assessed, as it heterodimerizes with NKG2A/C subunits during NK cell activation. There was no difference in the number of CD94+ cells between treatments and no significant differences were found in the expression levels of CD94-mid and -hi cells. The expression of CD49b, a common marker for NK identification, similarly exhibited no significant differences in expression between treatment groups. Thus, NK cells isolated from multiple compartments of a mouse model of COPD were phenotypically indistinguishable from the FA-exposed controls.

### Example 7

### NK Cell Cytotoxicity Against MHC Class I-Deficient Targets in a Mouse Model of COPD

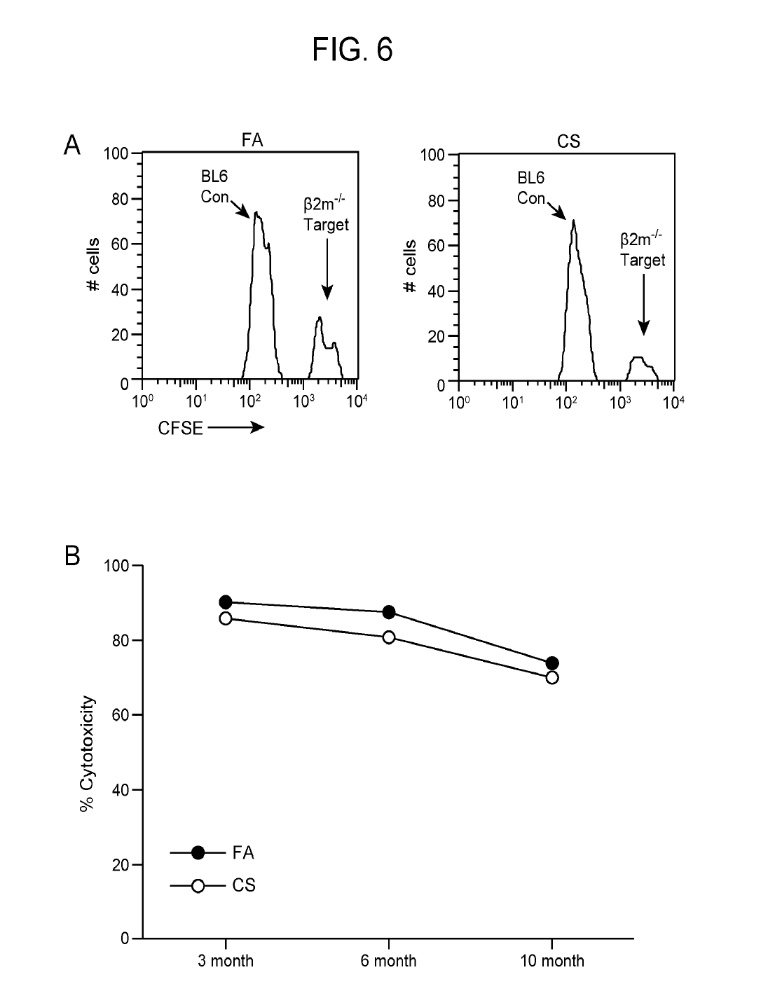

CS exposure increases CD107a expression (a marker of degranulation) on NK cells. In the current study, we specifically examined the function of NK cell cytotoxic activity in a mouse model of COPD. Utilizing an in vivo model of NK cell cytotoxicity, splenocytes from C57BL/6 wild-type and B2 m^{-/-} mice, which lack surface MHC class I antigen, were differentially labeled with the fluorescent marker carboxyfluorescein succinimidyl ester (CFSE) and injected i.v. The ratios of peripheral blood WT cells and B2 m^{-/-} cells recovered after the assay define NK cell cytotoxic effector function (FIG. 6, Panel A). NK cell cytotoxicity towards MHC class I-deficient cells was found not to be different in the COPD model compared to mice exposed to FA for the same duration (FIG. 6, Panel B). Notably, a similar significant decrease in cytotoxicity in mice exposed to CS or FA for 10 months was observed, indicating an age dependent decrease in baseline NK cell function (FIG. 6, Panel B).

### Example 8

### CS Exposure Enhances NKG2D-Mediated Cytotoxicity

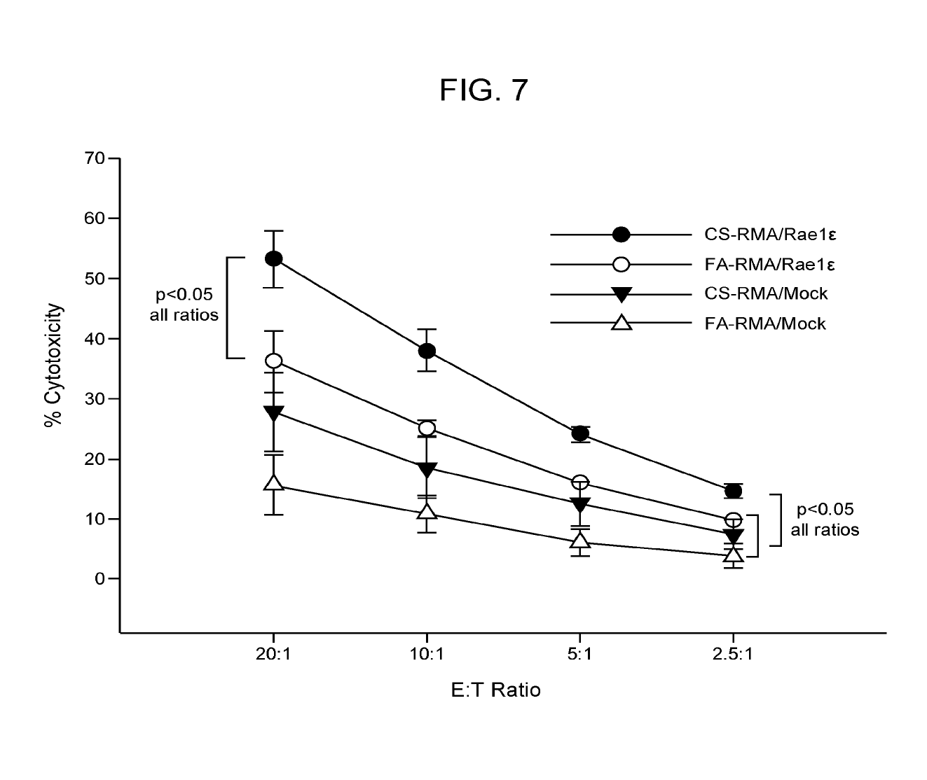

Engagement of the NKG2D (Klrk1) receptor is a potentially important pathway for NK cell activation in the context of COPD. Previously, it was demonstrated that NKG2D ligand expression is induced on airway epithelial cells of smokers and COPD patients and the alveolar and airway epithelium of mice exposed to CS. However, the specific function of NKG2D in COPD has not been examined. To investigate NKG2D function on NK cells, a cytotoxicity assay was performed using an RMA mouse T lymphoma cell line transfected to express Raet1ε. RMA cells tend to aggregate in organs making detection in blood difficult. Therefore, an ex vivo cytotoxicity assay was used to measure NK activity. As shown in FIG. 7, NK cells from mice exposed to CS were more cytotoxic towards RMA-Raet1ε cells than mice exposed to FA. The finding that NKG2D function is enhanced in the COPD model is significant because it identifies a unique mechanism whereby CS-exposure amplifies subsequent NK cell responses towards infected cells.

### Example 9

### NKG2D Deletion Abolishes NK Cell Hyperresponsiveness in Mouse Model of COPD

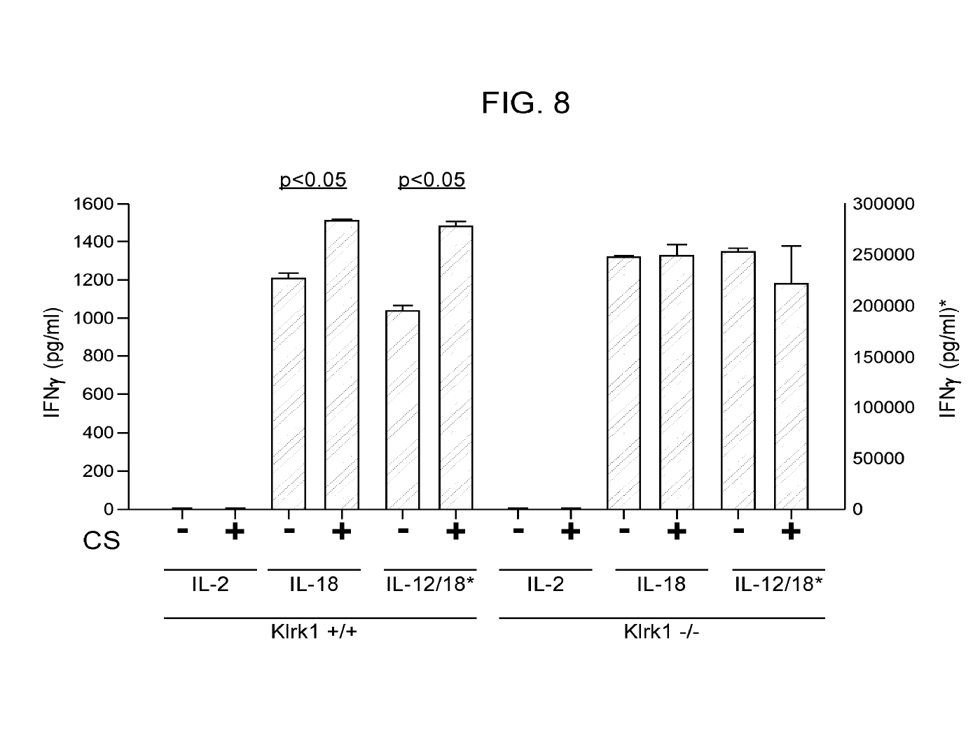

NK cell hypenesponsiveness in the context of CS exposure and in an inducible, lung-specific Raet1 expressing transgenic mouse model have been previously reported. To investigate the role of NKG2D in mediating CS-induced NK cell hyperresponsiveness, mice deficient in NKG2D (Klrk1^{-/-}) were utilized. NK cells were purified (˜99%) from FA and CS-exposed mice and stimulated with IL-18 or IL-12/IL-18. Consistent with previous data, CS exposure enhances NK cell IFNγ production after stimulation with cytokines (FIG. 8). The increased IFNγ production occurred absent significant differences in IL-12 or IL-18 receptor expression on NK cells between FA- and CS-exposed mice (data not shown). In contrast, NKG2D-deficient mice failed to exhibit increased cytokine responsiveness in a mouse model of COPD. Together with the current results demonstrating enhanced NKG2D function in our CS model of COPD (FIG. 7), these findings show that a hyperresponsive NK cell phenotype may be achieved through chronic, lung-specific NKG2D stimulation.

### Example 10

### NKG2D on NK Cells is Necessary for Enhanced Pulmonary Inflammation and Airway Injury Following Influenza Infection in COPD

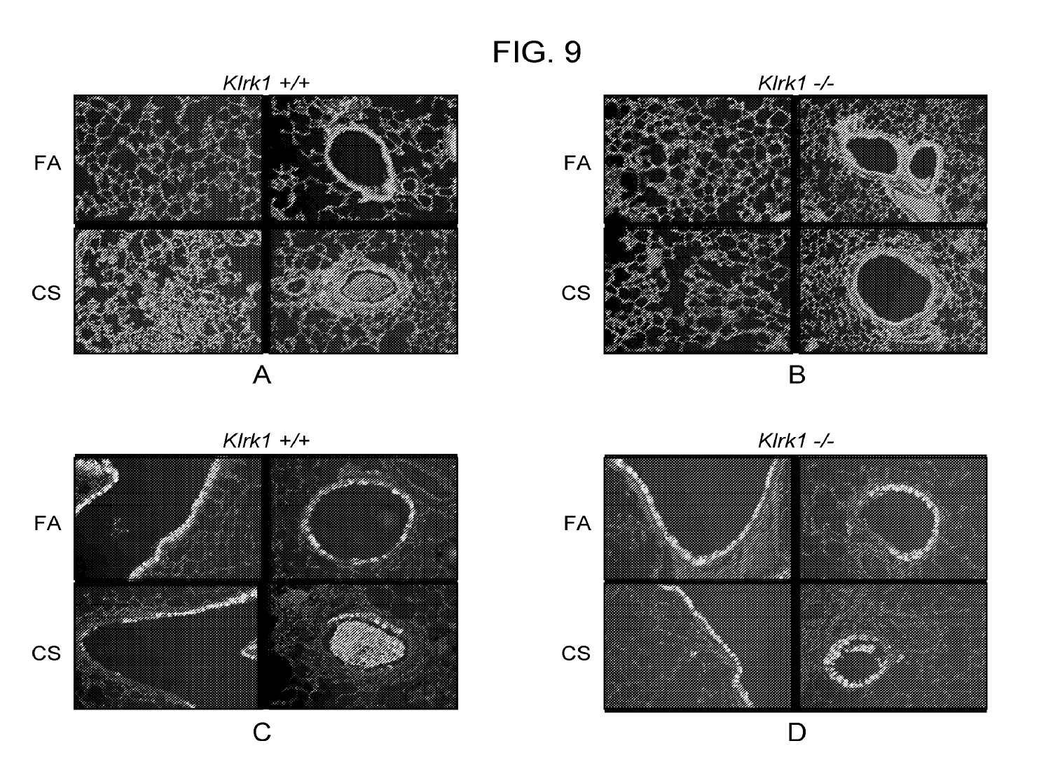

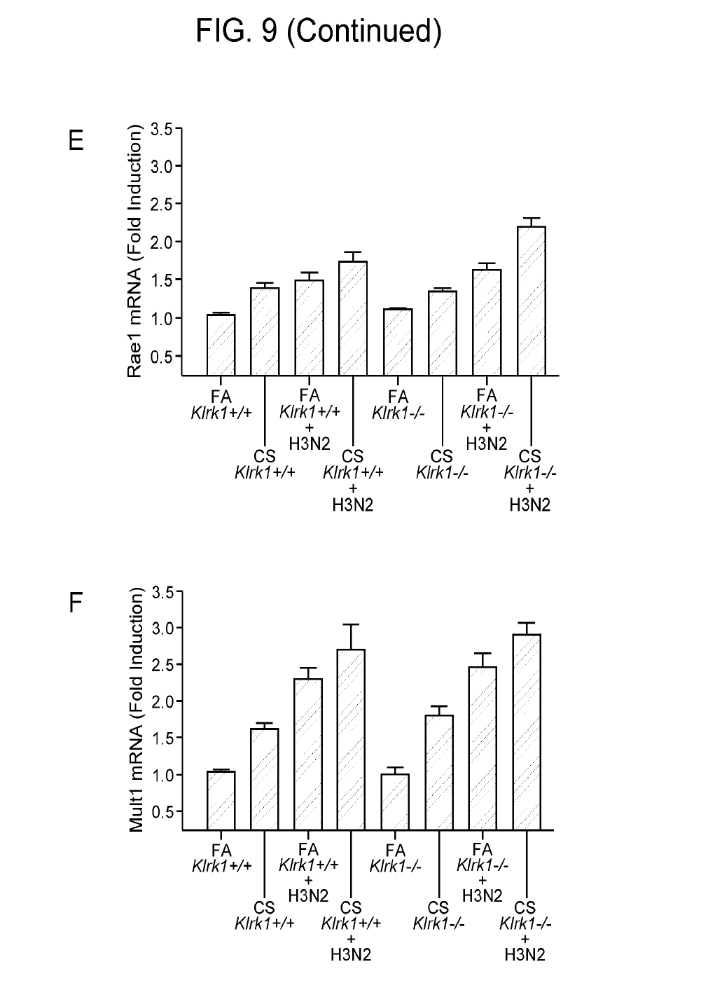

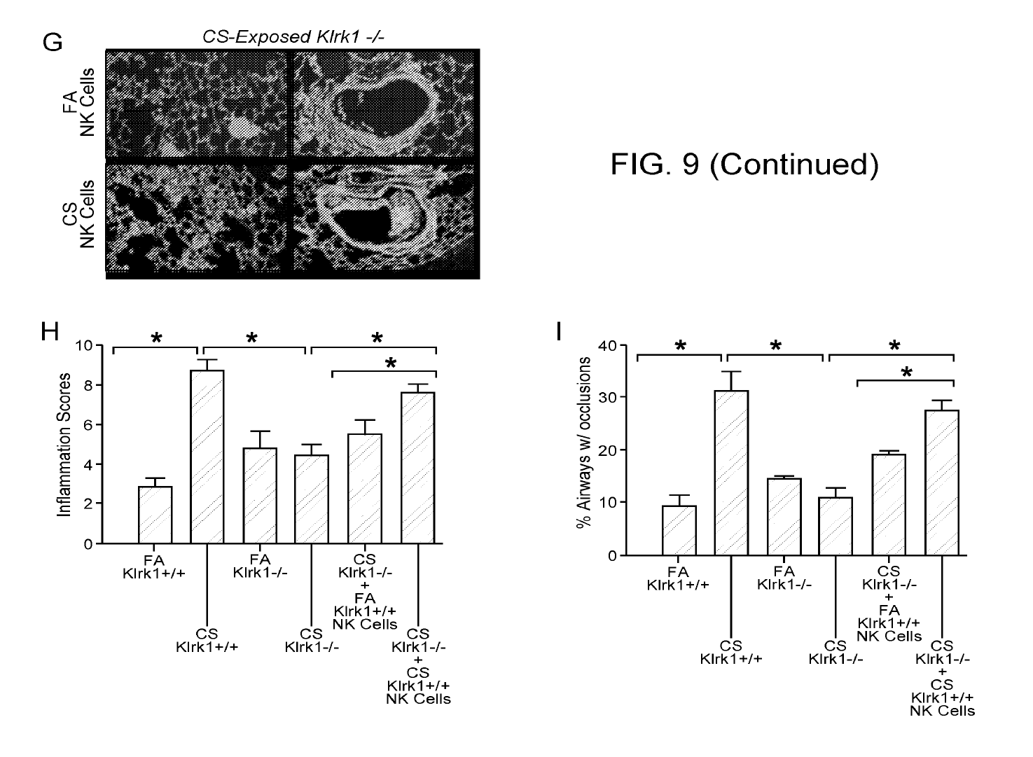

Viral infections contribute significantly to the pathogenesis of COPD. The above findings that NKG2D is required for NK cell hyperresponsiveness led us to examine the role of NKG2D in influenza-induced exacerbations of COPD. The early effects (i.e. 4 days post infection) of influenza infection were examined in our COPD model. Compared to influenza-infected mice exposed to FA, infection of mice exposed to CS augmented inflammation and airway epithelial injury that included both loss of epithelial integrity and differentiated epithelial cell morphology (FIG. 9, Panels A, C). Furthermore these exaggerated pathologies were found to be reduced in mice lacking the NKG2D receptor (FIG. 9, Panels B, D).

By staining with antibodies against Clara cell secretory protein (CCSP), a specific marker for airway epithelial cells, the extent of damage to the airway epithelium was assessed. CCSP staining demonstrated increased disruption of epithelial integrity within the large airways of CS-exposed Klrk1^{+/+} mice and that surviving epithelial cells had reduced expression of CCSP as a marker of epithelial differentiation relative to FA-infected Klrk1^{+/+} mice (FIG. 9, Panel C). In contrast, the damage to the airway epithelium of Klrk1^{-/-} mice was similar to FA-exposed Klrk1^{+/+} mice regardless of exposure (FIG. 9, Panel D). Similarly, influenza infection caused blockage of many of the small airways of CS-exposed Klrk1^{+/+} mice by large aggregates of cells that appeared to be a mix of denuded epithelial cells and leukocytes (FIG. 9, Panels A, C). These clumps of cells were infrequently observed in the FA-exposed Klrk1^{+/+} mice. Such mixed aggregates of cells/cellular debris were infrequently seen in the influenza infected Klrk1^{-/-} mice regardless of exposure (FIG. 9, Panels B, D).

To verify that the lack of the exaggerated response to influenza in the CS-Klrk1^{-/-} was attributable to receptor function and not the lack of NKG2D ligand induction, the affects of CS and influenza infection on transcript levels of multiple NKG2D ligands were assessed. As previously reported, CS exposure induces RAE1 expression in lung tissue. Here, CS exposure and influenza infections similarly upregulated Raet1 expression. An analogous pattern of Raet1 expression occurred in CS-exposed and influenza-infected Klrk1^{+/+} and Klrk1^{-/-} mice (FIG. 9, Panel E). Another NKG2D ligand, Mult1, demonstrated similar expression patterns to CS and influenza in both Klrk1^{+/+} and Klrk1^{-/-} mice (FIG. 9, Panel F).

The role of NKG2D specifically on NK cells in the exaggerated pathologies associated with influenza infection and COPD was sought to be defined. Purified lung NK cells from FA and CS-exposed Klrk1^{+/+} mice were transferred into CS-exposed Klrk1^{-/-} recipients which were then infected with influenza. Histopathological assessments were conducted to calculate inflammation severity and distribution as well as calculate the extent of airway obstruction. Influenza-infected Klrk1^{+/+} mice exposed to FA for 6 months and influenza-infected Klrk1^{-/-} mice exposed to FA or CS for 6 months exhibited significantly less inflammation than Klrk1^{+/+} CS-exposed mice (FIG. 9, Panels H, I). Transfer of NK cells from CS-exposed Klrk1^{+/+} donors into CS-exposed Klrk1^{-/-} recipients nearly recapitulated the phenotype of influenza infected CS-exposed Klrk1^{+/+} mice (compare FIG. 9, Panels G and A). The pathologies elicited with the transfer of NK cells from CS-exposed donors was also significantly greater than the pathologies associated with the transfer of NK cells from FA-exposed donors into the CS-exposed Klrk1^{-/-} recipients (FIG. 9, Panels H, I). These experiments demonstrate that NKG2D+NK cells from CS-exposed mice play a major role in inflammation and airway obstruction in the mouse model of COPD viral exacerbation.

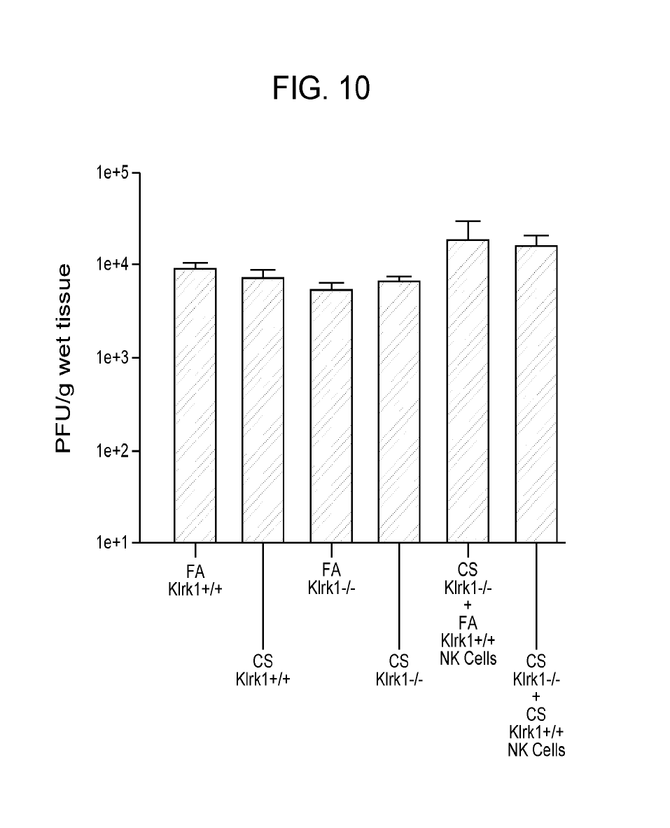

In addition to the inflammatory and histopathologic assays, the viral titers in the lungs of influenza-infected mice were assessed (FIG. 10). There were no significant differences between FA- and CS-exposed Klrk1^{+/+} and Klrk1^{-/-} mice or CS-exposed Klrk1^{-/-} mice that received Klrk1^{+/+} FA or CS NK cells. These studies demonstrate that NKG2D is necessary for the enhanced pulmonary inflammation and airway injury after influenza infection in COPD independent of the ability of the immune system to eliminate the virus.

Although the foregoing invention has been described in some detail by way of illustration and example for purposes of clarity of understanding, it is readily apparent to those of ordinary skill in the art in light of the teachings of this disclosure that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of treating or preventing a respiratory condition in a subject, said method comprising:
administering to the subject a composition comprising an inhibitor of NKG2D-mediated activation of leukocytes; and
a pharmaceutically acceptable vehicle, wherein the composition is administered in an amount effective to reduce or prevent symptoms of the respiratory condition in the subject.

2. The method of claim 1, wherein the respiratory condition is asthma or COPD.

3. The method of claim 1, wherein the inhibitor is an antibody or fragment thereof that binds to at least one of NKG2D and an NKG2D ligand.

4. The method of claim 3, wherein the antibody or fragment thereof increases cellular internalization of NKG2D in a leukocyte comprising surface-exposed NKG2D.

5. The method of claim 1, wherein the administering results in a reduction in IgE level in the serum of the subject.

6. The method of claim 1, wherein the inhibitor reduces expression of NKG2D in the subject.

7. A method of treating or preventing a respiratory condition in a subject suffering from or at a significant risk of developing the respiratory condition, said method comprising:
administering to the subject a composition comprising an inhibitor of NKG2D-mediated activation of leukocytes; and
a pharmaceutically acceptable vehicle, wherein the composition is administered in an amount effective to reduce or prevent symptoms of the respiratory condition in the subject.

8. The method of claim 7, wherein the respiratory condition is asthma or COPD.

9. The method of claim 7, wherein the inhibitor is an antibody or fragment thereof that binds to at least one of NKG2D and an NKG2D ligand.

10. The method of claim 7, wherein the administering results in a reduction in the number of leukocytes in the lung tissue of the subject.

11. The method of claim 7, wherein the inhibitor reduces expression of NKG2D in the subject.

12. A pharmaceutical composition comprising (a) a first respiratory condition treatment comprising an inhibitor of NKG2D-mediated activation of leukocytes as an active agent and (b) a second respiratory condition treatment active agent.

13. The pharmaceutical composition of claim 12, wherein the second respiratory condition treatment active agent is selected from bronchodilators, inhaled corticosteroids, leukotriene modifiers, long-acting beta agonists, combination inhalers, theophylline, immunomodulators, short-acting beta agonists, intravenous corticosteroids, phosphodiesterase-4 (PDE4) inhibitors, and expectorants.

14. A kit for treating a respiratory condition comprising:
(a) a composition comprising an effective amount of an inhibitor of NKG2D-mediated activation of leukocytes as an active agent; and
(b) instructions for using the composition to treat a respiratory condition in a subject in need thereof.

15. The kit according to claim 14, further comprising an effective amount of a second active agent for treating the respiratory condition.
